(19) United States
(12) Patent Application Publication
(10) Pub. No.: US 2008/0026049 A1
(43) Pub. Date: Jan. 31, 2008

(54) LIPOSOMAL COMPOSITIONS FOR PARENTERAL DELIVERY OF AGENTS

(76) Inventors: Ellen Wasan, Richmond (CA); Marcel Bally, Bowen Island (CA); Sebastian Cogswell, Vancouver (CA); Stuart Berger, Toronto (CA)

Correspondence Address: BERESKIN AND PARR, 40 KING STREET WEST, BOX 401, TORONTO, ON, M5H 3Y2, CA

(21) Appl. No.: 11/832,214

(22) Filed: Aug. 1, 2007

Related U.S. Application Data
(63) Continuation-in-part of application No. PCT/CA2006/000114, filed on Jan. 30, 2006.
(60) Provisional application No. 60/647,419, filed on Jan. 28, 2005.

(51) Int. Cl. A61K 9/127; A61K 31/235; A61K 31/35; A61K 31/351; A61K 31/40; A61P 13/12; A61P 31/00; A61P 9/00; A61P 19/00; A61K 31/416; A61K 31/4164; A61K 31/47; A61K 31/505

(52) U.S. Cl. 424/450; 514/275; 514/311; 514/399; 514/415; 514/423; 514/460; 514/533

(57) ABSTRACT

The invention provides methods and compositions for loading an agent onto a liposome for parenteral delivery. The methods are suitable for the loading of poorly soluble agents onto liposomes.

### CROSS-REFERENCE TO RELATED APPLICATIONS

This application is a continuation-in-part of PCT patent application number PCT/CA2006/000114, filed on Jan. 30, 2006 and published as WO 2006/079216, which claims the benefit of U.S. provisional application No. 60/647,419, filed Jan. 28, 2005, both of which are hereby incorporated by reference.

### FIELD OF THE INVENTION

The invention is, in general, in the field of drug delivery. More specifically, the invention provides methods and compositions for parenteral delivery of an agent, using a liposome delivery vehicle.

### BACKGROUND OF THE INVENTION

Liposomes are microscopic particles that are made up of one or more lipid bilayers enclosing an internal compartment. Liposomes have been widely studied and used as carriers for a variety of agents such as drugs, cosmetics, diagnostic reagents, and genetic material. Since liposomes consist of non-toxic lipids, they generally have low toxicity and therefore are useful in a variety of pharmaceutical applications. In particular, liposomes are useful for increasing the circulation lifetime of agents that have a short half-life in the bloodstream. Liposome-encapsulated agents often have biodistributions and toxicities which differ greatly from those of free agent. For specific in vivo delivery, the sizes, charges and surface properties of these carriers can be changed by varying the preparation methods and by tailoring the lipid makeup of the carrier. For instance, liposomes may be made to release an agent more quickly by decreasing the acyl chain length of a lipid making up the carrier.

Agents can be encapsulated in liposomes using a variety of methods and include membrane partitioning, passive encapsulation and active encapsulation. Agents that have hydrophobic attributes can intercalate into the lipid bilayer and this can be achieved by adding the agent during the liposome manufacturing process or by adding the agent to pre-formed liposomes. Agent encapsulation is often limited due to the ability of the liposome membrane to stably incorporate the agent. In addition the agent may adversely impact the physical properties of the liposomes. This method is also limited because the associated agent can rapidly transfer out of the membrane.

Passive loading involves the use of water-soluble or lipid soluble agents which are added to liposome components during the manufacturing process of liposomes. Some of the added agent will be encapsulated in the aqueous core or lipid bilayer of the liposomes and free agent (agent that has not been trapped within the liposome) can be removed by several standard separation methods. This procedure typically results in low trapping efficiencies and low agent to lipid ratios and is, therefore, not ideal.

Active loading techniques have been used to achieve high concentrations of agent in liposomes. Active loading involves the creation of pH gradients (ΔpH) or metal ion gradients (ΔM²⁺) across the liposomal bilayer. For example, a ΔpH generated by preparing liposomes in citrate buffer pH 4.0, followed by exchange of external buffer with buffered-saline pH 7.5, can promote the liposomal accumulation of weakly basic agent. The neutral form of the agent passively diffuses across the lipid bilayer and becomes trapped upon protonation in the low pH environment of the liposome interior. This process can result in >98% agent encapsulation and high agent-to-lipid ratios (e.g. vinorelbine, doxorubicin, vincristine, daunorubicin, mitoxantrone, to name a few). However, successful loading and retention using a transmembrane pH gradient is realized while the internal pH of the liposome is maintained. Since the pH gradient can dissipate following agent loading and since maintenance of the pH gradient is critical to achieve optimal agent retention, clinical formulation of pH gradient loaded agents requires the generation of a pH gradient across the liposomes just prior to agent loading or the use of formulations that maintain the pH gradient effectively after loading (e.g. use of strong buffers or ionophores that induce pH gradient formation). A second disadvantage of this method results from instability of lipid, and some agents, at acidic pH which decreases the long-term storage potential of the agent loaded liposomes. Freezing of liposomal formulations slows the rate of hydrolysis but conventional liposomal formulations often aggregate and leak contents upon thawing unless appropriately selected cryoprotectants are used.

Agent loading via ΔM²⁺ follows a process analogous to the pH gradient process, with agent accumulation being driven by metal ion-complexation (e.g. doxorubicin-Mn²⁺). Agent loading efficiencies are comparable to those described for the ΔpH process. However, loading efficiency is dependent on the choice of metal ion and agent.

Statins are a class of compounds that act as competitive inhibitors of hydroxymethyl-glutaryl (HMG)-CoA reductase. HMG-CoA reductase catalyzes the conversion of HMG-CoA to mevalonate, a rate limiting step in cholesterol biosynthesis. Inhibition of this enzyme decreases de novo cholesterol synthesis, increasing expression of low-density lipoprotein (LDL) receptors on hepatocytes. This increases LDL uptake TO by the hepatocytes, decreasing the amount of LDL cholesterol in the blood. Statins also reduce the blood levels of triglycerides and slightly increase levels of HDL-cholesterol. Accordingly, statins are known hypolipidemic agents and are used to lower cholesterol and prevent cardiovascular disease. Statins also appear to have anti-inflammatory effects that cannot be accounted for by their lipid lowering abilities. These include suppression of proinflammatory cytokine and chemokine production, immunomodulation and down regulation of endothelial cell activation (Blanco-Colio et al. [2003] Kidney Int. 63:12; Leung et al. [2003] J. Immunol. 170:1524). As a consequence of these properties, statin therapy has been examined in a number of chronic immune mediated inflammatory diseases including experimental autoimmune encephalomyelitis and arthritis, in particular rheumatoid arthritis (RA). The statin simvastatin has been shown to exhibit a therapeutic effect in the collagen induced arthritis (CIA) model of RA (Leung et al. [2003] J. Immunol. 170:1524). Atorvastatin was found to have a therapeutic effect in patients with RA as well as beneficially influencing inflammatory markers (McCarey et al. [2004] Nature, 363:2015). Other recent studies provide further support for the therapeutic effect of statins in patients with RA as well as other inflammatory disorders or conditions such as transplantation, multiple sclerosis, and chronic kidney disease (see for example: Connor et al. [2006] Arthritis Res. Ther. 8:R94; Kinderlerer et al. [2006] Arthritis Res. Ther. 8:R130; Steffens et al. [2006] Nat. Clin. Pract. Nephrol. 2:378; Jansen [2006] Rheumatology, 45:1577; Xu et al. [2006] Arthritis Rheum. 54:3441; Yamagata et al. [2007] Rheumatol. Int. 27:631; Davignon et al. [2005] Vase. Health Risk Manag. 1:29; Gazi et al. [2007] Clin. Exp. Rheumatol. 25:102; Okamoto et al. [2007] J. Rheumatol. 34:964; Haruna [2007] Arthritis Rheum. 56:1827). Other recent reports have suggested the use of statins in the prophylaxis and treatment of influenza (see for example: Enserink [2005] Science, 309:1976; Fedson [2006] Clin. Infect. Dis. 43:199; Rainsford [2006] Inflammopharmacology, 14:2; Terblanche [2006] Crit. Care, 10:168; Frost et al. [2007] Chest, 131:1006).

### SUMMARY OF THE INVENTION

The invention provides methods for loading an agent onto a liposome for parenteral delivery, compositions prepared using the methods, and uses thereof.

In one aspect, the invention provides a method for loading an agent into a liposome by combining the agent with a micelle-forming compound to form a micelle including the agent, where the agent is releasable from the micelle-forming compound, and adding the micelle to the liposome, where the micelle combines with the liposome such that the agent is loaded into the liposome to form a loaded liposome.

In alternative embodiments, the micelle may combine with the lipid bilayer of the liposome such that the micelle components, including the agent is incorporated into the outer leaflet or both inner and outer leaflets of the lipid bilayer of the liposome; the micelle-forming compound may include a hydrophilic or amphipathic moiety such as a PEG-lipid conjugate (e.g., DSPE-PEG₂₀₀₀)

In alternative embodiments, the agent may be dissolved in a solvent, such as ethanol. In alternative embodiments, the agent may be a compound that is poorly soluble. In alternative embodiments, the agent may be a therapeutic agent (e.g., econazole, an anticancer agent, an antifungal agent or a statin).

In alternative embodiments, the loaded liposome may be about 100 nm to about 200 nm in diameter. In alternative embodiments, the loaded liposome may be a unilamellar liposome. In alternative embodiments, the loaded liposome may include one or more of a lipid selected from DMPC or DPPC. In alternative embodiments, the loaded liposome may include a targeting agent.

In alternative aspects, the invention provides a composition produced by a method of the invention. In alternative embodiments, the composition may further include a pharmaceutically acceptable carrier.

In alternative aspects, the invention provides a liposomal composition including econazole, where the composition is formulated for parenteral delivery. In alternative embodiments, the composition may further include a lipid selected from DMPC or DPPC. In alternative embodiments, the composition may further include DSPE-PEG₂₀₀₀.

In alternative aspects, the invention provides a liposomal composition including a statin, where the composition is formulated for parenteral delivery. In alternative embodiments, the composition may further include a lipid selected from DMPC or DPPC. In alternative embodiments, the composition may further include DSPE-PEG₂₀₀₀.

In alternative aspects, the invention provides a method of treating a cancer or a fungal infection comprising administering a composition of the invention to a subject in need thereof. In alternative aspects, the invention provides the use of a composition of the invention for preparation of a medicament for treating a cancer or a fungal infection in a subject in need thereof. In alternative aspects, the invention provides the use of a composition of the invention for treating a cancer or a fungal infection in a subject in need thereof. In alternative aspects, the invention provides a method of delivering a therapeutic agent to a cell in a subject in need thereof by administering the composition of the invention to the subject.

In alternative aspects, the invention provides a method of treating a disease or condition that benefits from administration of a statin comprising administering a composition of the invention to a subject in need thereof. In alternative aspects, the invention provides the use of a composition of the invention for preparation of a medicament for treating a disease or condition that benefits from administration of a statin in a subject in need thereof. In alternative aspects, the invention provides the use of a composition of the invention for treating a disease or condition that benefits from administration of a statin in a subject in need thereof.

In alternative aspects, the invention provides a method for selecting a liposome composition having a desired loading or retention property for an agent, by preparing a first liposome composition by combining a vesicle-forming lipid with the agent under conditions suitable for forming a liposome such that the agent is loaded into the liposome; preparing a second liposome composition by combining the agent with a micelle-forming compound to form a micelle including the therapeutic agent, where the agent is releasable from the micelle-forming compound; adding the micelle to a liposome, where the micelle combines with the liposome such that the agent is loaded into the liposome; determining the amount of agent loaded onto the liposome or retained in the liposome in the first liposome composition and the second liposome composition, where a greater amount of agent loaded onto the liposome or retained in the liposome in the second liposome composition indicates a liposome composition having a desired loading or retention property in vitro or in vivo for the agent.

In alternative aspects, the invention provides a kit for preparing a loaded liposome including a first container including a therapeutic agent solubilized in a micelle and a second container including a liposome of the desired composition, together with instructions for combining the contents of the first and second containers to prepare a loaded liposome.

In alternative aspects, the invention provides a kit for preparing a loaded liposome including a first container including a therapeutic agent; a second container including a micelle-forming compound; and a third container including a liposome of the desired composition, together with instructions for combining the contents of the first and second containers to form a micelle including the therapeutic agent, and for combining the micelle with the contents of the third container to prepare a loaded liposome. In alternative embodiments, the therapeutic agent may be econazole or a statin; the micelle may include DSPE-PEG₂₀₀₀; and/or the liposome may include a lipid selected from DMPC or DPPC.

### BRIEF DESCRIPTION OF THE DRAWINGS

FIG. 1 depicts the chemical structure of econazole: [(dichloro-2,4 phenyl)-2(chloro-4 benzoyloxy)-2 ethyl]-1 imidazole nitrate.

FIG. 2 depicts the chemical structure of simvastatin, atorvastatin, cerivastatin, fluvastatin, lovastatin, mevastatin, pitavastatin, pravastatin and rosuvastin.

FIGS. 3A-B are schematic diagrams of the formulations. Symbols: curved lines: DSPE-PEG. Triangles: econazole. A. DSPE-PEG micelles added externally to liposomes containing econazole in the bilayer; B. DSPE-PEG/econazole micelles added to the outer leaflet of preformed liposomes.

FIGS. 4A-B are graphs demonstrating the drug to lipid ratio for during micelle exchange at 50° C. A: DMPC/DSPE-PEG (95:5 mol:mol); B: DPPC/DSPE-PEG (95:5 mol:mol). Diamonds (--⋄--) represent data for thin film/extrusion method of incorporating econazole into the liposomes and squares (-▪-) represent data for liposomal econazole prepared by the micelle exchange method. Data represent mean ±SD for 3 separate experiments within which each measurement was also performed in triplicate.

FIGS. 5A-B are bar graphs demonstrating the stability of liposomal econazole after 3, 10 or 20 days at 4° C. in HEPES buffered 150 mM NaCl (pH 7.2). A: DMPC/DSPE-PEG (95:5 mol:mol); A: DPPC/DSPE-PEG (95:5 mol:mol). Black bars: thin film/extrusion method of incorporating econazole into the liposomes; White bars: micelle-loading method. Data represent mean ±SD (n=3).

FIGS. 6A-D are graphs demonstrating the stability of micelle-loaded liposomal econazole. Liposomal econazole was incubated in HEPES-buffered saline (pH 7.2) or human plasma for 30 min at 37° C., followed by fractionation by gel filtration chromatography into liposome, micelle and protein-containing fractions. A and B represent the fractional distribution of DMPC/DSPE-PEG (95:5 mol:mol) formulations; C and D represent DPPC/DSPE-PEG (95:5 mol:mol) formulations. A and C show liposome components and B and D show econazole and protein fractional distribution. Black symbols represent samples that were incubated in buffer, while open symbols represent samples that were incubated in plasma. Symbols: Circles: liposomal lipid; squares: DSPE-PEG₂₀₀₀, triangles: econazole, diamonds: total protein (shown on B only for clarity). Data are mean ±SD, n=3 separate liposome preparations)

FIGS. 7A-B are graphs demonstrating the plasma elimination profile of liposomal econazole. Points represent 6 mice per timepoint (mean econazole concentration ±SD). A: Econazole elimination from plasma. B: drug to lipid ratio (w/w) vs. time

FIGS. 8A-B are graphs demonstrating the efficacy of liposomal econazole against MCF-7 tumors grown as xenografts in immunocompromised Rag2M mice. A: Treatment with liposomal econazole composed of DPPC/DSPE-PEG (95:5 mol/mol, micelle-loaded method) at 50 mg/kg or empty liposome vehicle control on days 17, 20, 22, 24, 27 and 29 (Indicated as ↑ on graph), starting when tumors were approximately 50 mm³. Data represent mean ±SEM (n=6 for vehicle controls and untreated controls, and n=5 for liposomal econazole treatment group). B: Data represents mean ±SEM for each treatment group (L-Econ: liposomal econazole; VC: vehicle control; UC: untreated control) for days 41-51 to illustrate the trend in controlling tumor growth for the liposomal econazole treatment group.

FIG. 9A-B are graphs showing the stability of MLV simvastatin and ML liposomal simvastatin in HBS buffer.

FIG. 10A-B are graphs showing the stability of MLV simvastatin and ML liposomal simvastatin in human plasma.

FIG. 11A-B are graphs showing the stability of MLV simvastatin and ML liposomal simvastatin in human RA synovial fluid.

FIG. 12 is a bar graph showing the drug:lipid ratio following of incubation of MLV simvastatin or ML liposomal simvastatin in HBS buffer and human synovial fluid.

FIGS. 13A-I are bar graphs showing the drug content of simvastatin-loaded liposomes with varying lipid compositions prepared using the standard thin film extrusion method (TFE) compared with the micelle loading method (ML) after incubation in HBS buffer

### DETAILED DESCRIPTION OF THE INVENTION

The present invention provides, in part, liposomal compositions for parenteral delivery of an agent (e.g., a therapeutic agent), and methods of preparation thereof. In some embodiments, the invention provides methods for increasing the concentration of poorly soluble agents (e.g., hydrophobic compounds) that can be achieved in liposomes. In some embodiments, the invention provides methods for increased incorporation of poorly soluble agents into liposomes. In some embodiments, such methods may: reduce the amount of a solvent required to solubilize a poorly soluble agent; or may extend the stability of liposomes containing a poorly soluble agent in the bloodstream of a subject; or may extend the stability of liposomes containing a poorly soluble agent during storage; or may increase the retention of a poorly soluble agent within a liposome during storage or in circulation in the bloodstream of a subject; or may otherwise improve the properties of a liposome containing a poorly soluble agent generally, either in vitro or in vivo. In some embodiments, use of a micelle as a means to solubilize a poorly soluble agent to be incorporated into a liposome increases the amount of that agent that can be stably incorporated into the liposome bilayer.

Liposomes

The term “liposome” as used herein means a vesicle including one or more concentrically ordered lipid bilayer(s) encapsulating an aqueous phase, when in an aqueous environment. Formation of such vesicles requires the presence of “vesicle-forming lipids” which are defined herein as amphipathic lipids capable of either forming or being incorporated into a bilayer structure. The term includes lipids that are capable of forming a bilayer by themselves or when in combination with another lipid or lipids. An amphipathic lipid is incorporated into a lipid bilayer by having its hydrophobic moiety in contact with the interior, hydrophobic region of the bilayer membrane and its polar head moiety oriented towards an outer, polar surface of the membrane. Hydrophilicity arises from the presence of functional groups such as hydroxyl, phosphate, carboxyl, sulfate, amino or sulfhydryl groups. Hydrophobicity results from the presence of a long chain of aliphatic hydrocarbon groups.

Liposomes can be categorized into multilamellar vesicles, multivesicular liposomes, unilamellar vesicles and giant liposomes. Multilamellar liposomes (also known as multilamellar vesicles or “MLV”) contain multiple concentric bilayers within each liposome particle, resembling the “layers of an onion”. Multivesicular liposomes consist of lipid membranes enclosing multiple non-concentric aqueous chambers. Unilamellar liposomes enclose a single internal aqueous compartment. Single bilayer (or substantially single bilayer) liposomes include small unilamellar vesicles (SUV) and large unilamellar vesicles (LUV). LUVs and SUVs range in size from about 50 to 500 nm and 20 to 50 nm respectively. Giant liposomes typically range in size from 5000 nm to 50,000 nm and are used mainly for studying mechanochemical and interactive features of lipid bilayer vesicles in vitro (Needham et al. [2000] Colloids and Surfaces B: Biointerfaces, 18: 183-195).

Any suitable vesicle-forming lipid may be utilized in the practice of this invention as judged by one of skill in the art. This includes phospholipids such as phosphatidylcholine (PC), phosphatidylglycerol (PG), phosphatidylinositol (PI), phosphatidic acid (PA), phosphatidyethanolamine (PE) and phosphatidylserine (PS); sterols such as cholesterol; glycolipids; sphingolipids such as sphingosine, ceramides, sphingomyelin, and glycosphingolipids (such as cerebrosides and gangliosides). Suitable phospholipids may include one or two acyl chains having any number of carbon atoms, between about 6 to about 24 carbon atoms, selected independently of one another and with varying degrees of unsaturation. Thus, combinations of phospholipid of different species and different chain lengths in varying ratios may be selected. Mixtures of lipids in suitable ratios, as judged by one of skill in the art, may also be used.

Liposomes for use in the present invention may be generated using a variety of conventional techniques. These techniques include: the ether injection method (Deamer et al., Acad. Sci.[1978] 308:250); the surfactant method (Brunner et al., [1976] Biochim. Biophys. Acta, 455:322); the Ca²⁺ fusion method (Paphadjopoulos et al., [1975] Biochim. Biphys. Acta, 394:483); the freeze-thaw method (Pick et al., [1981] Arach. Biochim. Biophys., 212:186); the reverse-phase evaporation method (Szoka et al., [1980] Biochim. Biophys. Acta, 601:559); the ultrasonic treatment method (Huang et al. [1969] Biochemistry, 8:344); the ethanol injection method (Kremer et al. [1977] Biochemistry, 16:3932); the extrusion method (Hope et al., [1985] Biochimica et Biophysica Acta, 812:55); the French press method (Barenholz et al., [1979] FEBS Lett., 99:210); or any other technique described herein or known in the art.

Different techniques may be appropriate depending on the type of liposome desired. For example, small unilamellar vesicles (SUVs) can be prepared by the ultrasonic treatment method, the ethanol injection method, or the French press method, while multilamellar vesicles (MLVs) can be prepared by the reverse-phase evaporation method or by the simple addition of water to a lipid film followed by dispersal by mechanical agitation (Bangham et al., [1965] J. Mol. Biol. 13:238-252). LUVs may be prepared by the ether injection method, the surfactant method, the Ca²⁺ fusion method, the freeze-thaw method, the reverse-phase evaporation method, the French press method or the extrusion method. In some embodiments, LUVs are prepared according to the extrusion method. The extrusion method involves first combining lipids in chloroform to give a desired molar ratio. A lipid marker may optionally be added to the lipid preparation. The resulting mixture is dried under a stream of nitrogen gas and placed in a vacuum pump until the solvent is substantially removed. The samples are then hydrated in an appropriate buffer or mixture of therapeutic agent or agents. The mixture is then passed through an extrusion apparatus (e.g. Extruder, Northern Lipids, Vancouver, BC) to obtain liposomes of a defined size. Average liposome size can be determined by quasi-elastic light scattering or photon correlation spectroscopy or dynamic light scattering or various electron microscopy techniques (such as negative staining transmission electron microscopy, freeze fracture electron microscopy or cryo-transmission electron microscopy). If desired, the resulting liposomes may be run down a Sephadex™ CL4B column or similar size exclusion chromatography column equilibrated with an appropriate buffer in order to remove unencapsulated drug or to create an ion gradient by exchange of the exterior buffer. Subsequent to generation of an ion gradient, LUVs may encapsulate therapeutic agents as set forth herein.

In some aspects, liposomes are prepared to be “cholesterol free”, meaning that such lipid-based vehicles contain “substantially no cholesterol,” or contain “essentially no cholesterol.” The term “cholesterol-free” as used herein with reference to a liposome means that the liposome is prepared in the absence of cholesterol, or contains substantially no cholesterol, or that the vehicle contains essentially no cholesterol. The term “substantially no cholesterol” allows for the presence of an amount of cholesterol that is insufficient to significantly alter the phase transition characteristics of the liposome (typically less than 20 mol % cholesterol). 20 mol % or more of cholesterol broadens the range of temperatures at which phase transition occurs, with phase transition disappearing at higher cholesterol levels. A liposome having substantially no cholesterol may have about 15 or less, or about 10 or less mol % cholesterol. The term “essentially no cholesterol” means about 5 or less mol %, or about 2 or less mol %, or about 1 or less mol % cholesterol. In some embodiments, no cholesterol will be present or added when preparing “cholesterol-free” liposomes. The presence or absence of cholesterol may influence the ability of the micelle-solubilized compound that can be stably incorporated into the liposome bilayer and may influence retention of that compound after incorporation.

Liposomes may range from any value between about 50 nm to about 1 nm in diameter. For example, liposomes in a liposomal composition according to the invention may range from any value between about 100 to about 140 nm in diameter. In some embodiments, liposomes in a liposomal composition according to the invention may be less than about 200 nm in diameter, or less than about 160 nm in diameter, or less than about 140 nm in diameter. In some embodiments, liposomes in a liposomal composition according to the invention may be substantially uniform in size, for example, 10% to 100%, or more generally at least 10%, 20%, 30%, 40%, 50, 55% or 60%, or at least 65%, 75%, 80%, 85%, 90%, or 95%, or as much as 96%, 97%, 98%, 99%, or 100% of the liposomes in the liposomal composition may be between the size values indicated herein. Liposomes may be sized by extrusion through a filter (e.g. a polycarbonate filter) having pores or passages of the desired diameter.

Liposomes may include a targeting agent (such as a sugar moiety, a cell receptor ligand, an antibody specific to a target cell, such as a cancer cell, a hepatocyte etc.) to achieve enhanced targeting to a specific cell population. Targeting agents may be incorporated into the surface of a liposome to optimize binding to target cells.

In some embodiments, liposomes may include a hydrophilic moiety. Grafting a hydrophilic moiety to the surface of liposomes can “sterically stabilize” liposomes thereby maximizing the circulation longevity of the liposome. This results in enhanced blood stability and increased circulation time, reduced uptake into healthy tissues, and increased delivery to disease sites such as solid tumors (see U.S. Pat. Nos. 5,013,556 and 5,593,622; and Patel et al., [1992] Crit Rev Ther Drug Carrier Syst, 9:39). Typically, the hydrophilic moiety is conjugated to a lipid component of the liposome, forming a hydrophilic polymer-lipid conjugate. The term “hydrophilic polymer-lipid conjugate” refers to a lipid, e.g., a vesicle-forming lipid, covalently joined at its polar head moiety to a hydrophilic polymer, and is typically made from a lipid that has a reactive functional group at the polar head moiety in order to attach the polymer. The covalent linkage may be releasable such that the polymer may dissociate from the lipid at for example physiological pH after a variable length of time, such as over several to many hours (Adlakha-Hutcheon et al. [1999] Nat Biotechnol. 17(8):775-9). Suitable reactive functional groups are for example, amino, hydroxyl, carboxyl or formyl groups. The lipid may be any lipid described in the art for use in such conjugates. The lipid may be a phospholipid having one or two acyl chains including between about 6 to about 24 carbon atoms in length with varying degrees of unsaturation.

In some embodiments, the lipid in the conjugate may be a PE, such as of the distearoyl form. The polymer may be a biocompatible polymer characterized by a solubility in water that permits polymer chains to effectively extend away from a liposome surface with sufficient flexibility that produces uniform surface coverage of a liposome. Such a polymer may be a polyalkylether, including polyethylene glycol (PEG), polymethylene glycol, polyhydroxy propylene glycol, polypropylene glycol, polylactic acid, polyglycolic acid, polyacrylic acid and copolymers thereof, as well as those disclosed in U.S. Pat. Nos. 5,013,556 and 5,395,619. The polymer may have an average molecular weight of any value between about 350 and about 10,000 daltons.

In alternative embodiments, the phospholipids may be selected from poly(ethylene glycol) (PEG) modified phospholipids. The average molecular weight of the PEG may be any value between about 500 to about 10,000 Daltons. Combinations of PEG phospholipid of different species and different chain lengths in varying ratios may be selected. Combinations of phospholipids and PEG phospholipids may also be selected. The conjugate may be prepared to include a releasable lipid-polymer linkage such as a peptide, ester, or disulfide linkage. The conjugate may also include a targeting agent. Mixtures of conjugates may be incorporated into liposomes for use in this invention.

In some embodiments, liposomes may include an agent, such as a therapeutic agent, prepared by conventional “active” or “passive” loading methods. For example, a therapeutic agent can be mixed with vesicle-forming lipids and be incorporated within a lipid film, such that when the liposome is generated, the therapeutic agent is incorporated or encapsulated into the liposome. Thus, if the therapeutic agent is substantially hydrophobic, it will be encapsulated in the bilayer of the liposome. Alternatively, if the therapeutic agent is substantially hydrophilic, it will be encapsulated in the aqueous interior of the liposome. The therapeutic agent may be soluble in aqueous buffer or aided with the use of detergents or ethanol. The liposomes can subsequently be purified, for example, through column chromatography or dialysis to remove any unincorporated therapeutic agent.

Liposomes may be prepared and formed in advance i.e., be “pre-formed” liposomes. Pre-formed liposomes may be used to prepare the liposomal formulations according to the invention. Such pre-formed liposomes may include an agent, such as a therapeutic agent, or an agent may be added to pre-formed liposomes prior to preparation of liposomal compositions according to the invention e.g., prior to combination with a micelle containing an agent. In some embodiments, pre-formed liposomes do not include a hydrophilic moiety. Pre-formed liposomes are available from various commercial contract pharmaceutical companies with expertise in the art of preparing liposomes.

Micelles

The term “micelle” as used herein means a self-assembled lipid arrangement without an internal aqueous phase and which generally has a mean diameter <50 nm. Micelles may be spherical or tubular or wormlike and form spontaneously at or above the critical micelle concentration (CMC). In general, micelles are in equilibrium with the monomers under a given set of physical conditions such as temperature, ionic environment, concentration, etc.

Formation of a micelle requires the presence of “micelle-forming compounds,” which include amphipathic lipids (e.g., a vesicle-forming lipid as described herein or known in the art), lipoproteins, detergents, non-lipid polymers, or any other compound capable of either forming or being incorporated into a micellar structure. Thus, a micelle-forming compound includes compounds that are capable of forming a monolayer by themselves or when in combination with another compound, and may be polymer micelles, block co-polymer micelles, polymer-lipid mixed micelles, or lipid micelles. A micelle-forming compound, in an aqueous environment, generally has a hydrophobic moiety in the interior of the micelle, and a polar head moiety oriented outwards into the aqueous environment. Hydrophilicity generally arises from the presence of functional groups such as hydroxyl, phosphate, carboxyl, sulfate, amino or sulfhydryl groups. Hydrophobicity generally results from the presence of a long chain of aliphatic hydrocarbon groups.

A micelle may be prepared from lipoproteins or artificial lipoproteins including low density lipoproteins, chylomicrons and high density lipoproteins. Artificial lipoproteins may also comprise lipidized protein with targeting capabilities. Uptake of lipoproteins into cell populations may be facilitated by receptors present on the target cells. For instance, uptake of low density lipoproteins into cancerous cells may be facilitated by LDL receptors present on such cells and uptake of chylomicrons and lactosylated high density lipoproteins into hepatocytes may be facilitated by the remnant receptor and the lactosylated receptor respectively.

Micelles for use in the present invention may be generated using a variety of conventional techniques. These techniques include: simple dispersion by mixing in aqueous or buffered or hydroalcoholic media or media containing surfactants or ionic substances; sonication, solvent dispersion or any other technique described herein or known in the art. Different techniques may be appropriate depending on the type of micelle desired and the physicochemical properties of the micelle-forming components, such as solubility, hydrophobicity and behaviour in ionic or surfactant-containing solutions.

Micelles for use in the present invention may range from any value between about 5 nm to about 50 nm in diameter. In some embodiments, micelles may be less than about 50 nm in diameter, or less than about 30 nm in diameter, or less than about 20 nm in diameter.

In some embodiments, micelles for use in the present invention may include a hydrophilic polymer-lipid conjugate, as described herein or known in the art. As indicated herein, the term “hydrophilic polymer-lipid conjugate” refers to a lipid, e.g., a vesicle-forming lipid, covalently joined at its polar head moiety to a hydrophilic polymer, and is typically made from a lipid that has a reactive functional group at the polar head moiety in order to attach the polymer. The covalent linkage may be releasable such that the polymer may dissociate from the lipid at for example physiological pH after a variable length of time, such as over several to many hours (Adlakha-Hutcheon et al. [1999] Nat Biotechnol. 17(8):775-9). Such conjugates may include any compounds known and routinely utilized in the art of sterically stabilized liposome technology and technologies which are useful for increasing circulatory half-life for proteins, including for example polyethylene glycol (PEG), polyvinyl alcohol, polylactic acid, polyglycolic acid, polyvinylpyrrolidone, polyacrylamide, polyglycerol, or synthetic lipids with polymeric head groups. For example, a distearoyl-phosphatidylethanolamine covalently bonded to a PEG alone, or in further combination with phosphatidylcholine (PC), may be used to produce a micelle according to the invention. The molecular weight of the PEG may be any value between about 500 Daltons to about 10,000 Daltons, inclusive, for example, 1000, 2000, 4000, 6000, 8000, etc. The CMC of the hydrophilic polymer-lipid conjugate will be dependent on the molecular weight of the PEG as well as the lipid anchor and the added components used when preparing mixed micelles (e.g. PEG modified distearoyl-phosphatidylethanolamine and PC).

Agents

Any active agent may be used in the liposomal compositions according to the invention. An “active agent” or “agent” or “compound” as used herein refers to a chemical moiety used in therapy or diagnosis, and includes any natural or synthetic biologically active agent, such as a peptide or polypeptide or analog thereof, a nucleic acid molecule or analog thereof, a small molecule, etc., and for which drug delivery in accordance with this invention is desirable. Thus, an agent includes therapeutic agents and imaging agents. The invention also encompasses, pharmaceutically acceptable salts, solvates and prodrugs of the active agent.

The term “pharmaceutically acceptable” means compatible with the treatment of patients.

The term “solvate” as used herein means an agent, or a salt of an agent, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of agents will vary depending on the agent and the solvate. In general, solvates are formed by dissolving a compound in the appropriate solvent and isolating the solvate by cooling or using an antisolvent. The solvate is typically dried or azeotroped under ambient conditions.

The term “pharmaceutically acceptable salt” includes both pharmaceutically acceptable acid addition salts and base addition salts.

The term “pharmaceutically acceptable acid addition salt” as used herein means any non-toxic organic or inorganic salt of any basic agent. Basic agents that may form an acid addition salt include, for example, those substituted with NH₂ NHC₁-C₂₀alkyl or N(C₁-C₂₀alkyl)(C₁-C₂₀alkyl). Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of statins are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the agents, for laboratory use, or for subsequent conversion to a pharmaceutically acceptable acid addition salt.

The term “pharmaceutically acceptable basic addition salt” as used herein means any non-toxic organic or inorganic base addition salt of any acid agent. Acidic agents that may form a basic addition salt include, for example, those possessing a carboxylic acid moiety. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art. Other non-pharmaceutically acceptable basic addition salts, may be used, for example, in the isolation of the agents, for laboratory use, or for subsequent conversion to a pharmaceutically acceptable basic addition salt.

The formation of a desired compound salt is achieved using standard techniques. For example, the neutral agent is treated with an acid or base in a suitable solvent and the formed salt is isolated by filtration, extraction or any other suitable method.

The term “prodrug” as used herein refers to any compound that has less intrinsic activity than the corresponding “drug,” but when administered to a biological system, generates the “drug” substance, either as a result of spontaneous chemical reaction or by enzyme catalyzed or metabolic reaction. Prodrugs include, without limitation, acyl esters, carbonates, phosphates, and urethanes. These groups are exemplary, and not exhaustive, and one skilled in the art could prepare other known varieties of prodrugs. Prodrugs may be, for example, formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

The agent or compound may be of any class which can be solubilized and incorporated into a micelle that includes micelle forming compounds. In alternative embodiments, the agent is “poorly soluble” in water or buffer, or under physiological conditions. A “poorly soluble” compound or agent is one that exhibits very low solubility, or is insoluble, in an aqueous environment, e.g., in an aqueous buffered solution at concentrations suitable for administration of pharmacologically relevant dosages of said compounds. In some embodiments, the term “poorly soluble” with reference to an active agent in water or buffer or physiological saline means that the active agent has a solubility in the water or buffer of less than about 10 mg/mL. Agent solubility can be measured and defined using standard techniques, for example, as indicated in the The United States Pharmacopoeia/The National Formulary standards and guidelines or other scientific reference manuals such as the Merck Index (Merck Co., Rahway, N.J.), or by any other means known in the art. For example, solubility of poorly soluble agents can be quantified based on octanol-water partition coefficient (LogP) or hydrophile-lipophile balance (HLB) scale (see for example Schott [1995] J Pharm Sci. 84(10):1215-22) and Schott [1984] J Pharm Sci. 73(6):790-2). In some embodiments, a poorly soluble agent exhibits a LogP of at least 1.5 or more. In some embodiments, a poorly soluble agent is one that is soluble in about 30 to about 10,000 or more parts of water for one part of solute, or from about 100 to about 1000 parts water/part solute, or from about 100 to about 10000 parts water/part solute, or from about 30 to about 100 parts water/part solute. The desired amount of agent to be incorporated into a liposome will depend in part on the potency of the agent where lower concentrations of a compound may be necessary for a potent agent. Poorly soluble agents include without limitation lipid soluble compounds, hydrophobic compounds, compounds poorly soluble at physiological pH, etc.

Any therapeutic agent (e.g., a poorly soluble agent) may be formulated in the liposomal compositions of the invention. Suitable therapeutic agents for use according to the methods of the invention include, without limitation, azole compounds, such as econazole, miconazole, and clotrimazole and statins, such as simvastatin, atorvastatin, cerivastatin, fluvastatin, lovastatin, mevastatin, pitavastatin, pravastatin and rosuvastin. Suitable therapeutic agents also include drugs such as Taxol® (paclitaxel), an etoposide-compound (etoposide and derivatives of etoposide with a similar core structure including teniposide), a camptothecin-compound (including topotecan, ironotecan, lurtotecan, 9-aminocamptothecin, 9-nitrocamptothecin and 10-hydroxycamptothecin, including salts thereof), a vinca-alkaloid or analog thereof, etc.

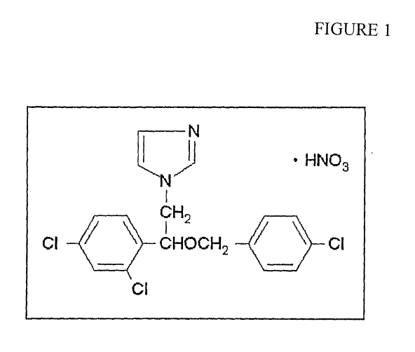

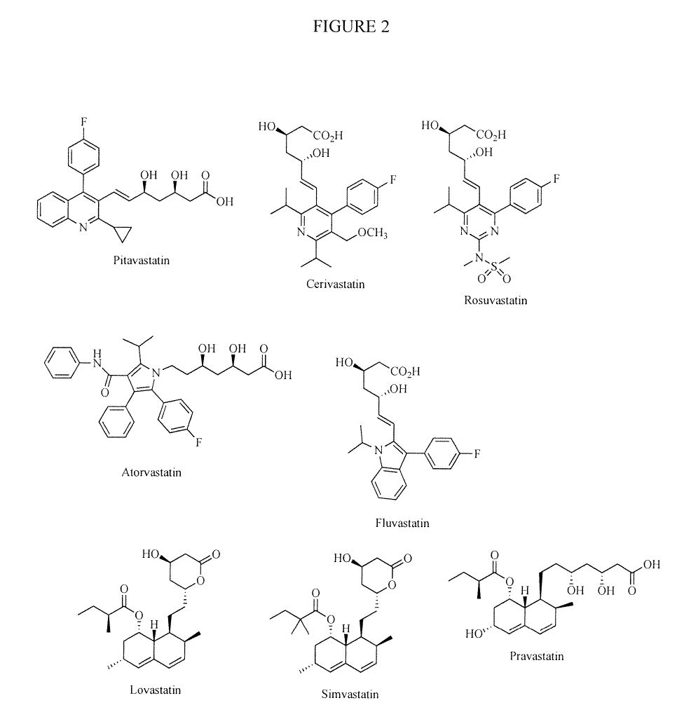

In one embodiment, a poorly soluble agent is an azole compound, such as econazole (FIG. 1), or a pharmaceutically acceptable salt, solvate or prodrug thereof, or mixtures thereof.

In another embodiment, a poorly soluble agent is a statin. In an embodiment, the statin is selected simvastatin, atorvastatin, cerivastatin, fluvastatin, lovastatin, mevastatin, pitavastatin, pravastatin and rosuvastin, mixtures thereof, pharmaceutically acceptable salts, solvates and prodrugs thereof and mixtures thereof. In an embodiment the statin is simvastatin or atorvastatin. In a further embodiment the statin is simvastatin. The chemical structure of simvastatin, atorvastatin, cerivastatin, fluvastatin, lovastatin, mevastatin, pitavastatin, pravastatin and rosuvastin is shown in FIG. 2.

Methods of Preparing Liposomal Compositions

The invention provides a method of preparing a liposomal composition including an agent or compound (e.g., a therapeutic agent such as econazole or a statin, which are used herein as model compounds) by incorporating the agent or compound into a micelle. The micelle may include a PEG-phospholipid, such as DSPE-PEG₂₀₀₀. The micelle is then combined with a liposome, such as a pre-formed liposome, thus incorporating the agent or compound into the liposome. In alternative embodiments, the agent is a poorly soluble compound that can be solubilized in a micelle. In alternative embodiments, liposomal compositions according to this invention are particularly suitable for the delivery of poorly soluble compounds or agents.

In some embodiments, the agent may be solubilized in a solvent, such as ethanol or hydroalcoholic solutions of ethanol in aqueous media, prior to incorporation into the micelle. In some embodiments, the final concentration of solvent in the phospholipid-containing liposomes, for example those composed primarily of DPPC, DMPC, DSPC, DOPC or similar compositions, may be limited to a concentration that does not induce significant toxicity when administered to a subject and/or does not disrupt the integrity or performance of the micelle or liposome. For example, for ethanol, the final concentration may be any value between about 1 to about 30% (v/v), although lower or higher values are also contemplated. In some embodiments, the incorporation of poorly soluble agents into liposomes can be achieved while minimizing solvent concentrations or the presence of bio-incompatible solvents. For agents to be encapsulated within the liposomal bilayer which are directly soluble in aqueous dispersions of the micelle-forming components, solvents such as ethanol may not be necessary.

A compound or agent may be incorporated into a micelle during preparation of a micelle as described herein or known in the art. For example the compound or agent may be dissolved in, for example, aqueous buffer/alcoholic media and combined with a buffer solution comprising the micelle forming compound and the resulting combination mixed and optionally warmed, for example to a temperature of about 30° C. to about 70° C., suitably about 55° C., until a substantially clear solution is obtained.

In alternative embodiments, the compound or agent is not covalently coupled to a micelle forming compound.

The agent-containing micelles are then incorporated into the liposomes, by, for example, combining the solution of the agent-containing micelles with a buffered solution containing the preformed liposomes and optionally warming to a temperature of about 30° C. to about 70° C., suitably about 35° C. to about 55° C., for up to 90 minutes. In an embodiment, the components of the micelles, including the agent, are incorporated into the outer leaflet or both leaflets of the bilayer of the liposomes. The liposomes may include but are not limited to one or more of the following lipids: DMPC, DPPC or DSPE, and the ratios of the lipids may vary according to embodiments visualized by persons skilled in the art of liposome preparation. In some embodiments, the liposome may be a pre-formed liposome that may or may not contain the therapeutic agent or one or more second or additional agent(s) (e.g., a small molecule, a protein, antibody, or polypeptide or a nucleic acid, e.g., having membrane localization properties such as juxtamembrane localization or transmembrane domains) incorporated or encapsulated in it. The second or additional agent may be loaded into the liposome using conventional loading techniques as described herein or known in the art. Alternatively, or additionally, more than one agent may be loaded into a liposome using the methods of the invention, by for example incorporating one or more micelles containing one or more agents into the liposome. In an alternative embodiment, small molecules (chemical compounds), proteins, antibodies or peptides or pharmaceutically acceptable salt thereof, may be encapsulated into a liposome by prior solubilization, active loading or passive entrapment and incorporation into a polymer micelles, polymer-lipid mixed micelles or lipid micelles.

Liposomal compositions according to the invention may be stored in any suitable form that may vary according to mode of administration. For example, a liposomal composition may be a liquid at room temperature (e.g., a sterile single-vial liquid), a frozen product, or a dehydrated product (e.g., a powder or a lyophilized cake to be reconstituted prior to use). Different storage forms may be prepared using methods known to a person skilled in the art. For example, a cryoprotectant such as a disaccharide, may be added to a liposomal composition prior to lyophilization to enable storage of a liposomal composition as a dehydrated product.

In alternative embodiments, the compound or agent is releasable from a liposome prepared according to the invention, to facilitate transfer of the compound or agent into a target cell. Thus, a releasable agent is an agent that is capable of transferring out of a liposome according to the invention and exerting its biological action inside, or in the vicinity of, a cell in a subject. In alternative embodiments, the compound or agent is generally stable during storage of a liposomal composition. In alternative embodiments, the compound or agent is generally stable during circulation in the bloodstream of a subject i.e., the compound or agent is not substantially released from the liposome prior to its delivery inside, or in the vicinity of, a cell in a subject.

As described herein, econazole or simvastatin PEG-lipid micelles including DSPE-PEG₂₀₀₀ were each added to pre-formed liposomes including DMPC or DPPC. Econazole and simvastatin were rapidly loaded and remained stably incorporated into the liposomes.

Therapeutic Indications

Liposomal compositions according to this invention may be used for delivery of a therapeutic agent, for example a poorly soluble therapeutic agent, for treatment of a variety of diseases and conditions in a subject in need thereof, or for bringing about a desired biological effect such as an immune response in such a subject. Such diseases and conditions include those that would benefit from liposomes which increase retention or stability of the therapeutic agent in storage or in circulation in a subject, enabling therapeutic drug interventions with superior ADMET (absorption, distribution, metabolism, excretion and toxicity) properties. Examples of therapeutic uses of the compositions of the present invention include treating cancer, treating cardiovascular diseases such as hypertension and those associated with elevated cholesterol levels, cardiac arrhythmia and restenosis, treating bacterial, viral, fungal or parasitic infections, treating and/or preventing diseases through the use of the compositions of the present inventions as vaccines, treating inflammation or treating autoimmune diseases.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. “Treating” or “treatment” as used herein includes prevention of a condition or disease, and accordingly, prophylactic uses of the liposomal compositions of the invention are also included within the scope of the invention.

By a “cancer” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumors, and carcinomas such as breast cell carcinomas and prostate carcinomas. The term cancer includes cell growths that are technically benign but which carry the risk of becoming malignant. By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor.

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to mestastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body, that is different from the primary site. Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas. Lymphomas may arise in the lymph nodes associated with the head, neck and chest, as well as in the abdominal lymph nodes or in the axillary or inguinal lymph nodes. The following list provides some non-limiting examples of primary cancers and their common sites for secondary spread (metastases):

| Primary cancer | Common sites for metastases |
|---|---|
| prostate | bone |
| breast | bone, lungs, skin, brain |
| lung | bone, brain |
| colon | liver, lungs, bone |
| kidney | lungs, bone |
| pancreas | liver, lungs, bone |
| melanoma | lungs |
| uterus | lungs, bones, ovaries |
| ovary | liver, lung |
| bladder | bone, lung |

Tumor vasculature is generally leakier than normal vasculature due to fenestrations or gaps in the endothelia. This may allow liposomes of about 200 nm in diameter or less to penetrate the discontinuous endothelial cell layer and underlying basement membrane surrounding the vessels supplying blood to a tumor. Selective accumulation of the delivery vehicles into tumor sites following extravasation leads to enhanced delivery and effectiveness of the therapeutic agent. In order to promote extravasation, targeting agents directed against tumor associated endothelial cells may be bound to the outer surface of the liposomes. In some embodiments, a targeting antibody may be covalently or non-covalently incorporated on the surface of the liposome to enable specific localization of the liposome to areas of disease; for example metastatic cancer cells which have spread to other sites in the body. In some embodiments, a therapeutic antibody may be incorporated into the liposome.

In an aspect of the invention the liposomal compositions are useful for the treatment of a disease or condition that benefits from administration of a statin. In embodiments of the invention, the disease or condition that benefits from administration of a statin is selected from dyslipidemia, hypercholesterolemia, hypertriglyceridemia, cardiovascular disease, acute coronary syndrome, experimental autoimmune encephalomyelitis, rheumatoid arthritis, osteoarthritis, transplantation, multiple sclerosis, chronic kidney disease and influenza. In an embodiment, the disease or condition is selected from dyslipidemia, hypercholesterolemia, hypertriglyceridemia, cardiovascular disease, acute coronary syndrome, rheumatoid arthritis and influenza. In a further embodiment, the disease or condition is selected from dyslipidemia, hypercholesterolemia, hypertriglyceridemia, cardiovascular disease and rheumatoid arthritis.

Statins, when taken in oral form, undergo extensive hepatic metabolism, reducing the amount of active agent that can get to the areas where they are needed (for example an arthritic joint). Further, a well-known side effect of statins is myopathy, or muscular weakness. Encapsulation of liposomes can reduce hepatic metabolism and penetration into muscle cells thus improving the therapeutic effects of this important class of compounds.

Pharmaceutical & Veterinary Compositions, Dosages, and Administration

In some embodiments, the compositions of the invention are particularly useful for the delivery of poorly soluble compounds. Compounds or agents in the liposomal compositions of the invention can be provided alone or in combination with other compounds or agents (for example, nucleic acid molecules, small molecules, peptides, or peptide analogues), in the presence any pharmaceutically acceptable carrier, in a form suitable for administration to mammals, for example, humans, pigs, horses, cattle, sheep, etc. In some embodiments, the compositions may include an adjuvant. In some embodiments, the liposomal compositions may include a targeting agent to localize or direct the liposomes to the region or tissue requiring exposure to therapeutic doses of the therapeutic agent. In some embodiments, the targeting agent may be an antibody or component that selectively recognizes a tumor or diseased cell or tissue. If desired, treatment with a liposomal composition according to the invention may be combined with more traditional and existing therapies for the condition to be treated. Compounds or agents according to the invention may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Conventional pharmaceutical practice may be employed to provide suitable formulations or compositions to administer the compounds to subjects suffering from or at risk for cancer, fungal infection, etc. In some embodiments, the pharmaceutical compositions are administered parenterally, i.e. intraarticularly, intravenously, subcutaneously, or intramuscularly or via aerosol. Aerosol administration methods include intranasal and pulmonary administration. In some embodiments, the pharmaceutical compositions are administered intravenously, intramuscularly or intraperitoneally by a bolus injection. For example, see Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; or Fountain et al., U.S. Pat. No. 4,588,578.

Methods well known in the art for making formulations are found in, for example, “Remington's Pharmaceutical Sciences (2003-20th edition) and in The United States Pharmacopeia: The National Formulary (USP 24 NF19) published in 1999. Formulations for parenteral administration may, for example, contain excipients, sterile water, or saline, polyalkylene glycols such as polyethylene glycol, oils of vegetable origin, or hydrogenated napthalenes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. In some embodiments, a liposomal composition according to the invention is not suitable for topical administration. In some embodiments, a liposomal composition according to the invention is particularly suitable for parenteral administration, e.g., by injection.

The liposomal compositions according to the invention are in general capable of delivering an effective amount of a compound or agent to a cell in a subject. An “effective amount” of a compound or agent according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a compound or agent may vary according to factors such as the disease state, age, sex, and weight of the individual, and the ability of the compound or agent to elicit a desired response in the individual. Dosage regimens may be adjusted to provide the optimum therapeutic response. A therapeutically effective amount is also one in which any toxic or detrimental effects of the compound or agent are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. A suitable range for therapeutically or prophylactically effective amounts of a compound may be any integer from 0.1 nM-0.1M, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) or agent(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in unit dose form for ease of administration and uniformity of dosage.

In the case of vaccine formulations, an immunogenically effective amount of a compound or agent can be provided, alone or in combination with other compounds or agents, with an immunological adjuvant, for example, Freund's incomplete adjuvant, dimethyldioctadecylammonium hydroxide, or aluminum hydroxide. The compound or agent may also be linked with a carrier molecule, such as bovine serum albumin or keyhole limpet hemocyanin to enhance immunogenicity.

In general, compounds, agents and compositions of the invention should be used without causing substantial toxicity. Toxicity of the compounds, agents and compositions of the invention can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be necessary to administer substantial excesses of the compositions.

The compositions may be administered to any suitable subject. As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for having a disorder, be diagnosed with a disorder or be a control subject that is confirmed to not have the specific disorder of interest.

Kits

The liposomal compositions of the invention may be provided in a kit, together with instructions for use. The kit may include a first container including an agent solubilized in a micelle, a second container including a liposome of a desired composition, and instructions for mixing the contents of the first and second containers at a desired ratio to provide a liposomal composition containing the agent (i.e., to provide a loaded liposome).

In alternative embodiments, the kit may include a first container including an agent; a second container including a micelle-forming compound; and a third container including a liposome of the desired composition, together with instructions for combining the contents of the first and second containers to form a micelle loaded with the agent, and for combining the micelle with the contents of the third container to prepare a loaded liposome containing the agent.

In some embodiments, the kit may include a second agent to be loaded into the liposome using convention techniques, prior to combining the liposome with a micelle.

The kit components may be stored at suitable temperatures or forms, e.g., room temperature, refrigerated (e.g., 4° C.), frozen (e.g., −20° C.), cryopreserved, dehydrated, etc., for suitable lengths of time.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

Various alternative embodiments and examples of the invention are described herein. These embodiments and examples are illustrative and should not be construed as limiting the scope of the invention.

### EXAMPLE 1

### Liposomal Formulation with Econazole

Materials

Econazole was purchased from Sigma-Aldrich (St. Louis, Mo. USA) as a nitrate salt powder. Dipalmitoyl phosphatidylcholine (DPPC), dimyristoyl phosphatidylcholine (DMPC) and distearoyl phosphatidylethanolamine-poly(ethylene glycol)₂₀₀₀ (DSPE-PEG) with an average PEG molecular weight of 2000 were purchased from Avanti Polar Lipids (Albaster, Ala.). Tritiated cholesteryl hexadecyl ether ([³H]-CHE) and [¹⁴C]-distearoyl phosphatidylethanolamine-poly(ethylene glycol) ([¹⁴C]-DSPE-PEG₂₀₀₀) were purchased from Perkin Elmer (Boston, Mass., USA). Whatman Nuclepore 200 nm, 100 nm or 80 nm filters were used in a 3 ml Lipex Extruder, all from Northern Lipids (Vancouver, B.C., Canada). Sephadex G-50 and Sepharose CL-4B size-exclusion chromatography beads were also purchased from Sigma. Other reagents were either from Sigma or Fisher Chemicals (Fairlawn, N.J., USA). All solvents were HPLC grade. Water was prepared by a reverse osmosis system (MilliQ) and filtered (0.22 μm) prior to use. Buffers were also filtered prior to use (0.22 μm).

Econazole UV Spectrophotometric Assay

Econazole was dissolved in methanol up to a concentration of 25 mg/ml and a characteristic absorption peak was discovered in the ultraviolet range (λ=271 nm). Econazole experimental samples were quantified by comparison with a standard curve (r²≧0.995) with a linear range of 0.05-1.0 mg/ml. For liposomal econazole, the absorbance readings of empty liposomes were subtracted from liposomal econazole samples as background, and samples were typically diluted at 1:10 (v/v) in methanol (to clarity) prior to analysis to solubilize the liposomes and econazole.

Liposome Preparation

Lipid constituents were weighed out in the desired mole to mole ratio and solubilized in chloroform. A nonexchangeable, nonmetabolized radioactive lipid tracer, [³H]-CHE (˜0.5 μCi/μmol) was added to the dissolved lipids for lipid quantitation post extrusion (Derksen, 1987). The lipid solution was dried to a thin film under N₂ gas, followed by hydration with HEPES-buffered saline (HBS: 25 mM HEPES, 150 mM NaCl, pH 7.2) at 50° C. for DPPC and 37° C. for DMPC for 1 h with frequent vortexing. Five cycles of freeze and thaw were then performed with liquid N₂ and a 37° C. waterbath. The sample was then extruded at 50° C. (DPPC) or 37° C. (DMPC) by passing the sample 10 times through two stacked polycarbonate filters of 200 nm pore size with a Lipex (Mayer 1986). Quasi-elastic light scattering (Nicomp 270, Particle Sizing Systems, Santa Barbara, Calif.) was used to determine mean diameter and particle size distribution of the liposomes and micelles (Table 1).

Particle size determined by quasi-elastic light scattering of liposomes immediately after extrusion through 2×200 nm filters and after the addition of DSPE-PEG micelles. Data represent mean ±SD (n=3 to 6 independent preparations).

Liposomal Formulations

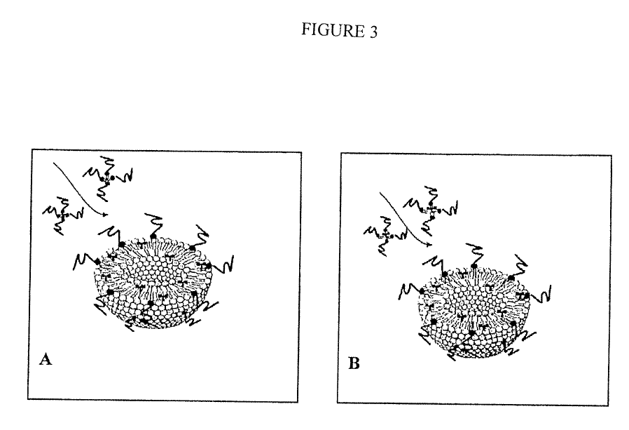

Econazole was incorporated into the lipid bilayer during liposome formation followed by exchange of DSPE-PEG₂₀₀₀ into the outer leaflet (FIG. 3A, thin-film/extrusion method), or econazole was incorporated into the outer leaflet of the lipid bilayer by exchange of DSPE-PEG/econazole micelles into pre-formed liposomes. (FIG. 3B, micelle-loading method) In the first case, DMPC or DPPC and econazole were mixed during the thin film stage of liposome preparation followed by extrusion, as described above. Separately, DSPE-PEG₂₀₀₀ was solubilized in HBS/ethanol (2:1 v/v), heated to 37° C. (DMPC) or 50° C. (DPPC) until clear micelles were formed (˜15 nm diameter) and then added to the warmed liposomes at 5 mol % and final ethanol 4.3% (v/v) (FIG. 3A). For the micelle-loading method, DMPC or DPPC liposomes were prepared first by extrusion as described above. Micelles of DSPE-PEG₂₀₀₀ in HBS/ethanol (2:1 v/v) also containing econazole were prepared by mixing and warming (50° C.) for approximately 30 min until clarity. By dynamic light scattering, the micelles formed were ˜15 nm diameter. Liposomes and micelles were then combined by mixing at 37° C. (DMPC) or 50° C. (DPPC) for the times indicated in the results section, up to 90 min. The final econazole concentration was 5 mg/mL and the lipid ratio (DMPC or DPPC: DSPE-PEG₂₀₀₀) was 95:5 (mol:mol). The ethanol concentration was 4.3% (v/v) upon first combining the liposomes and DSPE-PEG/econazole micelles.

Analysis of Drug Loading

Liposomes were incubated with the micelles of DSPE-PEG ±econazole for 5, 15, 30, 60 or 90 minutes at 37° C. (DMPC-containing liposomes) or 50° C. (DPPC-containing liposomes). To separate liposome-associated econazole from free or micelle-associated econazole, 100 μl of sample were added with 50 μl of HBS in triplicate at each timepoint to 1 mL size exclusion Sephadex G-50 columns, and centrifuged at 792×g for 2 min. The minicolumns were pre-equilibrated in HBS (pH 7.2). The liposome-containing eluate was analyzed by UV spectroscopy for econazole as described above. Lipid concentration was measured by triplicate scintillation counting of the [³H]-CHE lipid tracer, and the drug:lipid ratio (w/w) was calculated at each timepoint. For each sample type, at least three independent liposome preparations were analyzed, and the mean drug:lipid ratio at each time point is reported.

Stability Analyses

Stability testing was performed to observe how long the econazole would remain associated with the liposomes: a) in HBS at 4° C. and b) in the presence of human plasma at 37° C. For stability studies in buffer, liposomes were stored at 4° C. for 3, 10 or 20 days, then at each timepoint 100 μl of the sample were applied to mini Sephadex size exclusion columns in triplicate with 50 μl of HBS. The columns were centrifuged 792×g for 2 minutes and the elute was analyzed for lipid and econazole concentration as described above to determine the drug to lipid ratio.

Results

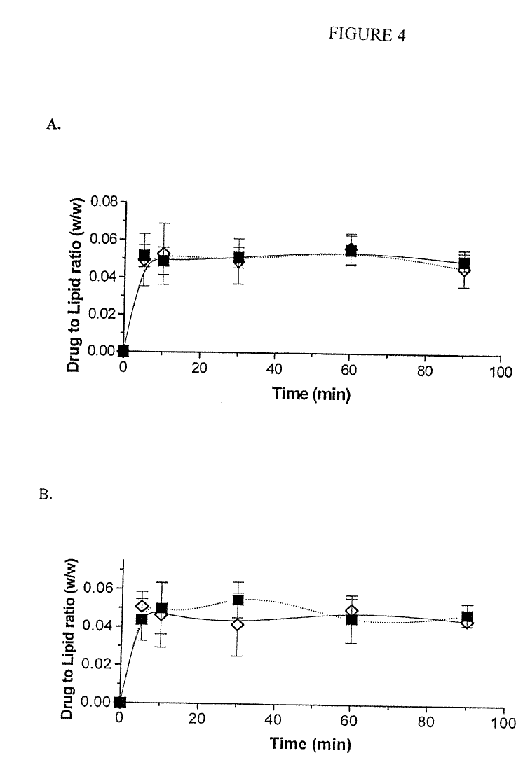

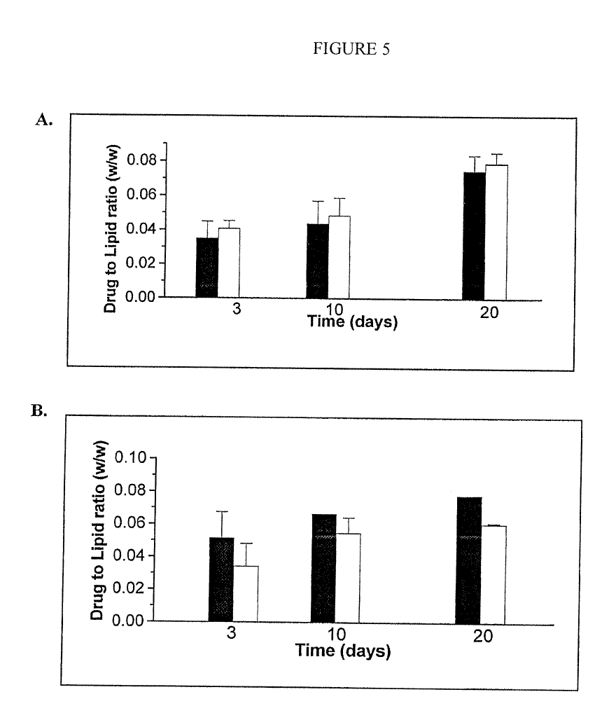

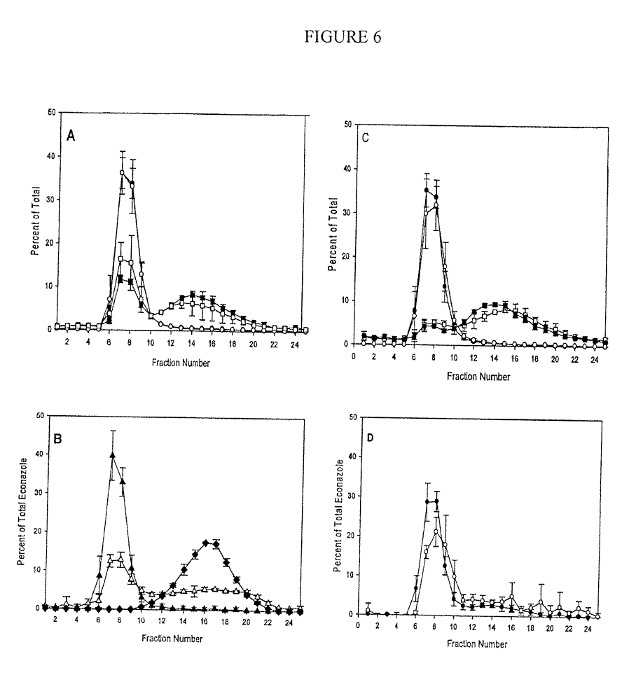

All liposomal formulations exhibited 100% drug loading at 0.05 drug:lipid ratio (w/w) of econazole (5 mg/mL). (FIG. 4) Significantly, the methods described here allowed for much easier hydration and extrusion steps than the formulations already containing PEG-lipid in the lipid film stage. Liposomes composed of 100% DPPC formed reversible aggregates quickly after extrusion. However, after the addition of DSPE-PEG₂₀₀₀ micelles and incubation at 50° C. for 30-60 min, a stable decrease in particle size and polydispersity indicated a reversal of the aggregation (Table 1). After the addition of DSPE-PEG₂₀₀₀ micelles with or without econazole the liposomal mean diameter was 140-160 nm. The lack of a significant separate particle population <50 nm is consistent with incorporation of the DSPE-PEG micelles or DSPE-PEG/econazole micelles into the liposomes. In the absence of PEG-lipid, control DPPC/econazole liposomes [100:5 (w/w)] were not stable and tended to aggregate within 2 hours. DMPC-based liposomes did not aggregate for any formulation step. Stability experiments showed that econazole remains stably associated with the liposomes for at least 3 weeks in HBS (pH 7.2) at 4° C. with no significant change from the 0.05 drug:lipid ratio originally loaded (FIG. 5) nor in particle size, with mean diameters remaining as in Table 1 throughout the study period.

### EXAMPLE 2

### Plasma Stability of Liposomal Econazole In Vitro

For stability studies in plasma, three separate preparations of micelle-loaded liposomal econazole were made with trace [¹⁴C]-CHE and [³H]-DSPE-PEG₂₀₀₀ as described above using DMPC or DPPC as the main lipid constituent. The liposomes were mixed with human plasma at a ratio of 1:3 (v/v) and incubated at 37° C. for 30 min. The plasma was applied to a 10 mL CL4B size exclusion chromatography column equilibrated in HBS and at least 25 fractions were collected at a rate of 0.7 ml/min to determine if econazole and PEG-lipid were associated with liposomes or with plasma protein-containing fractions. Each fraction was analyzed in triplicate for [¹⁴C]-CHE as a measure of the liposome-containing fractions, econazole, [³H]-DSPE-PEG₂₀₀₀ or total phosphate as a measure of PEG-lipid stability in the liposomes, and total protein. The three measures were averaged for each parameter, and these means were combined from the 3 different batches of liposomes for the data represented in the figures. Protein analysis was performed by visible spectrophotometry (λ=562 nm) using the bicinchoninic acid assay (Sigma) and compared to a triplicate standard curve of bovine serum albumin (linear range=0-100 μg/ml, r²≧0.995). The presence of empty liposomes, DSPE-PEG/econazole micelles or drug-loaded liposomes did not affect the fractional distribution of plasma proteins on the column. Likewise, the fractional distribution of the liposomes was not affected by the presence of econazole (in the liposomes or in DSPE-PEG micelles) or plasma proteins. Econazole analysis was by liquid-liquid extraction consisting of fraction sample, H₂O and ethyl acetate at a ratio of 1:1:6 (v/v/v). Samples were vortexed for 5 min and centrifuged at 10,000×g for 5 min. The top organic layer was removed, dried under N₂ gas and reconstituted in 100 μL methanol. The econazole assay was performed as described above. Background consisted of the corresponding extracted fractions of empty liposomes.

For the micelle-loaded liposomal econazole, stability in plasma was assessed by measuring drug:lipid ratio of the liposomes after incubation in plasma for 30 min at 37° C. Size exclusion chromatography was used to separate liposome-associated econazole from econazole associated with DSPE-PEG micelles or plasma proteins. For clarity of the figure, the liposome components and econazole are plotted in separate figures as percent of total component loaded onto the size exclusion columns. (FIG. 6) Approximately 49% of the econazole remained associated with the DPPC/DSPE-PEG liposomal fraction (FIG. 6, fractions 5-9) following the incubation period in plasma, compared to 95% for liposomes incubated in buffer. Approximately 34% was recovered in the partially overlapping protein and micelle fractions (FIG. 6, fractions 13-19) after incubation in plasma, compared to 2% in controls incubated in buffer. In the case of DMPC/DSPE-PEG/econazole micelle-loaded liposomes, 66% was recovered in the liposomal fraction after incubation in human plasma, compared to 81% in buffer, and 23% eluted in the protein/micelle fractions, compared to 13% eluting in those fractions after incubation in buffer. Due to the poor solubility of econazole in HBS (<0.1 mg/mL, near the limit of detection by the UV spectrophotometric assay) a separate free drug fraction was not detected upon elution from the column, but would likely have represented <1% of the total, based on mass balance of all collected fractions. Also of interest was the stability of the DSPE-PEG₂₀₀₀ in the liposomes. Approximately 37% of the DSPE-PEG was retained by the DPPC liposomal fraction after incubation in buffer and 50% after incubation in plasma, whereas in the DMPC-based liposomes, only 16% was retained in the liposomal fraction after incubation in buffer and only 20% after incubation in plasma. For this reason, the DPPC-based formulations were pursued in favor of the DMPC liposomes for the pharmacokinetic and efficacy studies, because the retention of PEG-lipid is presumed to be important in maximizing liposome circulation time and thereby tumor accumulation.

### EXAMPLE 3

### In Vivo Tolerability

Multidose Tolerability Studies in Mice

Single dose and multi-dose tolerability studies were performed on Rag2M female mice at 50 mg/kg econazole dose via intravenous injection into the lateral tail vein at a volume of 200 μl/20 g mouse once (single dose) or every other day for 6 doses (multidose). The care, housing and use of animals were performed in accordance with the Canadian Council on Animal Care Guidelines. Four formulations were tested in the single-dose study, comparing DPPC and DMPC liposomes containing econazole prepared by the thin film/extrusion method vs. the micelle-loaded form. In all cases the final lipid ratio was 95:5 (mol/mol) (DPPC or DMPC:DSPE-PEG₂₀₀) and the drug:lipid ratio was 0.05 (w/w). The vehicle controls consisted of the corresponding liposomes not containing econazole. For the multidose study, only the DPPC-based liposomal econazole formulations were assessed prior to efficacy studies, because their stability was greater than the DMPC-based liposomes.

For both the single and multi-dose studies, mice (n=3/group) were weighed daily during the drug administration period and for 14 days after the last dose. Observation of appearance and behavior also continued for 14 days after the last dose and scored by a certified animal technician to ascertain morbidity. At the end of the study, the mice were terminated by CO₂ inhalation and blood was collected immediately by cardiac puncture. The blood was allowed to clot for 1 hour, and then the serum was separated by centrifugation 1000×g for 15 min. Serum was frozen in liquid N₂ and stored at −20° C. until shipment to Central Laboratory for Veterinarians (Surrey, BC, Canada) for analysis of liver enzymes (alkaline phosphatase, AST, ALT, GGT, bilirubin, sorbital dehydrogenase), electrolytes, BUN and creatinine.

The single-dose tolerability study in Rag2M immunocompromised mice showed that the liposomal econazole formulations were all well tolerated at 50 mg/kg econazole dose [drug:lipid ratio=0.05 (w/w)] i.v. bolus with no obvious differences between treatment groups. The multidose tolerability study showed that DPPC-based liposomal econazole formulations were well tolerated at 50 mg/kg econazole [drug:lipid ratio=0.05 w/w)] i.v. bolus every other day excluding weekends for 6 doses. Serum was collected for analysis of liver enzymes (alkaline phosphatase, ALT, AST, GGT, bilirubin and sorbital dehydrogenase) in both the multidose tolerability study, at 14 days after the last of 6 doses, and in the efficacy study, at day 59 post tumor inoculation at the termination of the study (42 days after treatment stopped). In the multi-dose study, serum analysis indicated mild elevations in liver enzymes (ALT, GGT) in the liposomal econazole groups and less so in the vehicle control group (n=3 mice/group, lipid dose in all groups 1000 mg/kg) compared to the laboratory normal ranges for mice. (Table 2) Serum was collected 14 days after the last of 6 doses (50 mg/kg) i.v. every other day.

Serum was pooled from 2 mice to produce 3 samples of sufficient volume for analysis (n=6 mice/group). Data represent mean ±SD. Arrows indicate increase (↑) above the normal range for mice, which is indicated at the top of each column.

Table 3 indicates the results of serum analysis from the efficacy study, where elevations in alkaline phosphatase, AST and GGT were noted, with greater increases associated with the liposomal econazole loaded by the thin film method. Alkaline phosphatase was elevated in all groups receiving liposomes, and in groups receiving econazole, bilirubin was slightly elevated in 1 of 3 samples in both groups. Results of serum electrolyte analysis showed elevated potassium levels in all groups receiving liposomes, however, BUN and creatinine were not elevated. (Table 4) Necropsy revealed pale liver and kidneys in several animals in all groups of the multidose study and the efficacy study, including the vehicle control group, which is consistent with the relatively high lipid dose.

Serum was collected at day 59 post-tumor inoculation from mice bearing MCF-7 xenograft tumors. Treatment with liposomal econazole (50 mg/kg i.v. for 6 doses) occurred on days 17, 20, 22, 24, 27 and 29). Serum was pooled from 2 mice to produce 3 samples of sufficient volume for analysis (n=6 mice/group). Data represent mean ±SD. Arrows indicate increase (↑) above normal range for mice, with the number of mice exhibiting the change indicated (e.g. 2 out of 3 samples: 2/3). Normal ranges for mice are indicated at the top of each column in parentheses.

Serum was collected at day 59 post-tumor inoculation from mice bearing MCF-7 xenograft tumors. Treatment with liposomal econazole (50 mg/kg i.v. for 6 doses) occurred on days 17, 20, 22, 24, 27 and 29). Serum was pooled from 2 mice to produce 3 samples of sufficient volume for analysis (n=6 mice/group). Data represent mean ±SD. Arrows indicate increase (↑) above normal range for mice, which is indicated at the top of each column in parentheses.

### EXAMPLE 4

### Pharmacokinetics of Liposomal Econazole

Reverse-Phase HPLC Assay

For analysis of pharmacokinetic samples, 200 μl of plasma were extracted 2 times with 3 volumes of ethyl acetate and 2 volumes of 0.1M NaOH, with vortexing for 15 min for each extraction, and centrifugation at 1500×g for 10 min to separate organic and aqueous phases. The combined organic phases were dried at 60° C. under vacuum in a vortex-evaporator in approximately 20 min. The dried extract was reconstituted in 100-200 μL acetonitrile and centrifuged to remove any residue. The supernatant (10 μL) was injected onto the HPLC by autoinjector the same day. The HPLC column was a NovaPak RP-18 (C18, 75×46 mm, 4 μm) and the mobile phase was acetonitrile: 10 mM ammonium formate+20 mM diethylamine (64:36) run at a flow rate of 1 mL/min at 28° C. column temperature. UV detection (λ=270 nm) was performed with a photodiode array detector (Waters 996). Quantitation of samples was performed using an external standard curve of econazole prepared in triplicate in mouse plasma, using the same extraction method as the samples (r²>0.995, linear range: 20-250 μg/mL, limit of detection=10 μg/mL). Extraction efficiency, was ˜90% across the concentration range. Data analysis was performed using WinNonLin version 1.5 software (Scientific Consulting, Inc.,) and comparison of means was performed using MicroCal Origin software with two-way Anova, where significance was set at p=0.05.

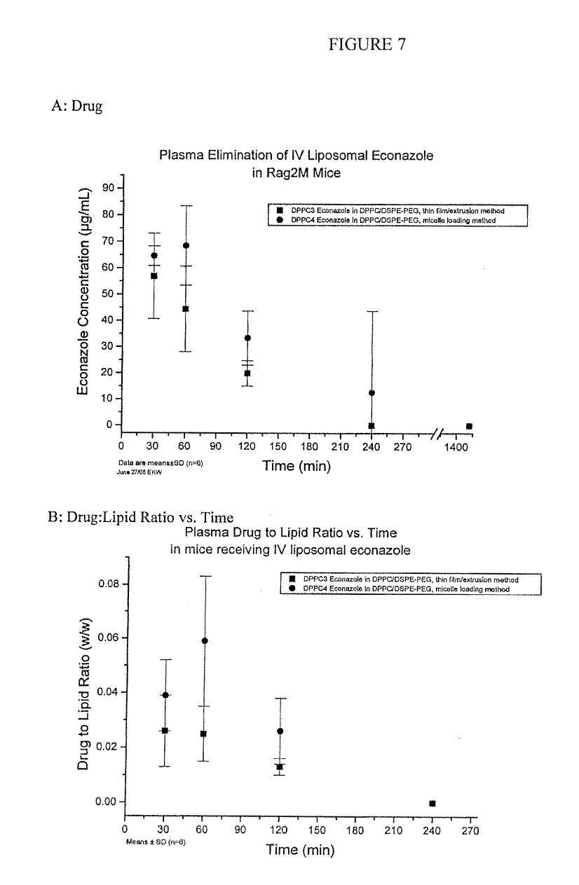

Rag2M mice were injected intravenously with liposomal econazole that was prepared by either the thin-film/extrusion method or by the micelle-loading method. Analysis of econazole concentration in the plasma vs. time (FIG. 7) showed that the majority of both formulations of liposomal econazole was cleared from the plasma by 2 hours and that elimination appears to follow a first-order elimination process. The area under the curve for the measured timepoints (AUC₀₋₂₄₀ₘᵢₙ) was estimated to be 196 mg/ml min for the thin-film/extrusion liposomal econazole and 281 mg/mL min for the micelle-loaded liposomal econazole and plasma half-life of approximately 30.9 min, and 34.3 min, respectively. The drug-to-lipid ratio was significantly different between the two formulations at 15, 30 and 60 minutes (p<0.05), with the micelle-loaded form showing a higher drug-to-lipid ratio at those timepoints.

### EXAMPLE 5

### Efficacy of Liposomal Econazole in MCF-7 Xenografts in Rag2M Mice

Mice received estradiol as 60-day slow-release subcutaneous pellets one day prior to tumor cell inoculation. The mice were injected with 1×10⁵ MCF-7 cells (American Type Culture Collection, ATCC) subcutaneously. The mice were injected with 200 μl/20 g of liposomal econazole or empty liposomes via the lateral tail vein once the tumors reached approximately 50 mm³, with dosing every other day excluding weekends for a total of 6 doses, starting at day 17 post-tumor inoculation. Tumors were measured daily until day 59, at which time the mice were sacrificed and serum was collected for analysis as described above. Observation of appearance and behavior also continued throughout the study period, scored by a certified animal technician to ascertain morbidity.

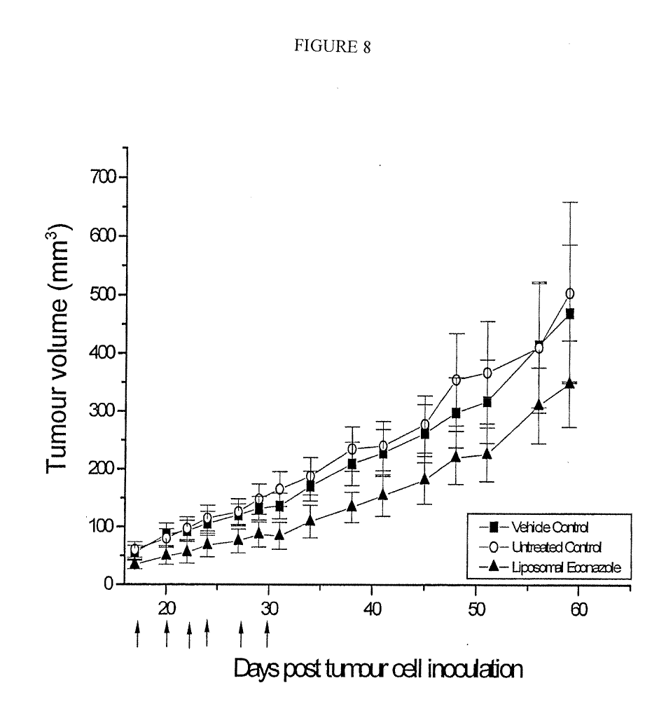

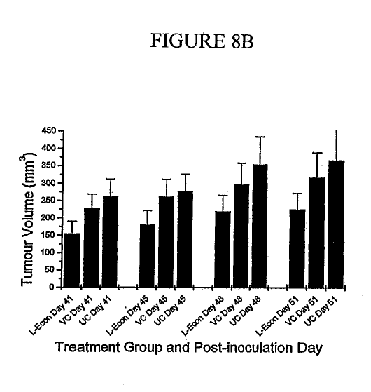

Liposomal econazole prepared using DPPC/DSPE-PEG₂₀₀₀ (95:5 mol/mol) by the micelle-loading and the thin-film/extrusion methods were chosen for in vivo testing because stability studies up to that point indicated that they would be more suitable than the DMPC-based formulations. Untreated controls and mice receiving empty liposomes (vehicle control) reached a tumor volume of 300 mm³ by day 48, whereas there was a 10-12 day tumor growth delay in the liposomal econazole groups. Mean ±SEM There was also a trend to reduced tumor volume growth of the liposomal econazole groups, which behaved similarly, was significantly less than compared to that of the vehicle control group and untreated control group (Anova, p<0.05) (FIG. 8). It should be noted that tumor growth was relatively controlled in the liposomal econazole group, resulting in a lower tumor volume two days after completion of the 6 doses (Day 31) compared to the control groups (FIG. 8 inset), and the growth rate did not increase until treatment stopped.

### EXAMPLE 6

### Liposomal Formulation with Simvastatin

Simvastatin (Toronto Research Chemicals, North York, Ontario Canada) was prepared at 2 mg/mL in liposomes composed of dipalmitoyl phosphatidyl choline/distearoyl phosphatidylethanolamine-poly(ethylene glycol)₂₀₀₀ (DPPC/DSPE-PEG₂₀₀₀ 95:5 mol:mol) by passively entrapping simvastatin in multilamellar vesicles (MLVs) or by incorporating the drug into large unilamellar vesicles (LUVs,) by the micelle loading (micelle exchange or ML) method described in Example 1.

### EXAMPLE 7

### Stability Studies for Liposomal Simvastatin in HBS Buffer, Human Plasma and Human Rheumatoid Arthritis (RA) Synovial Fluid

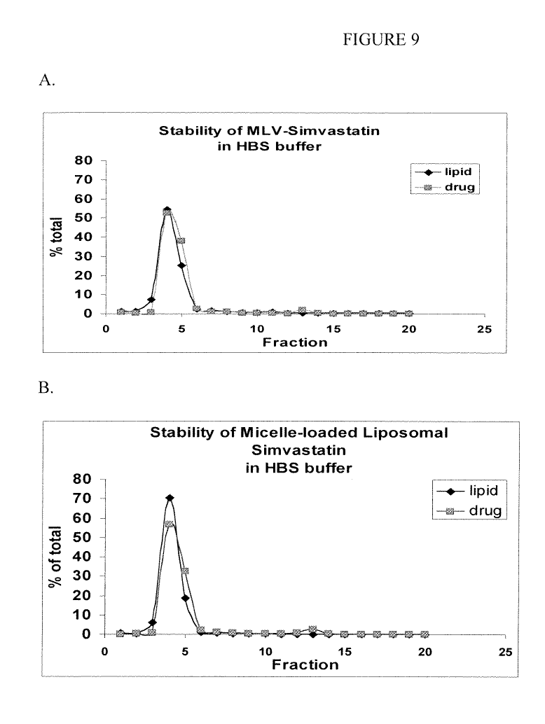

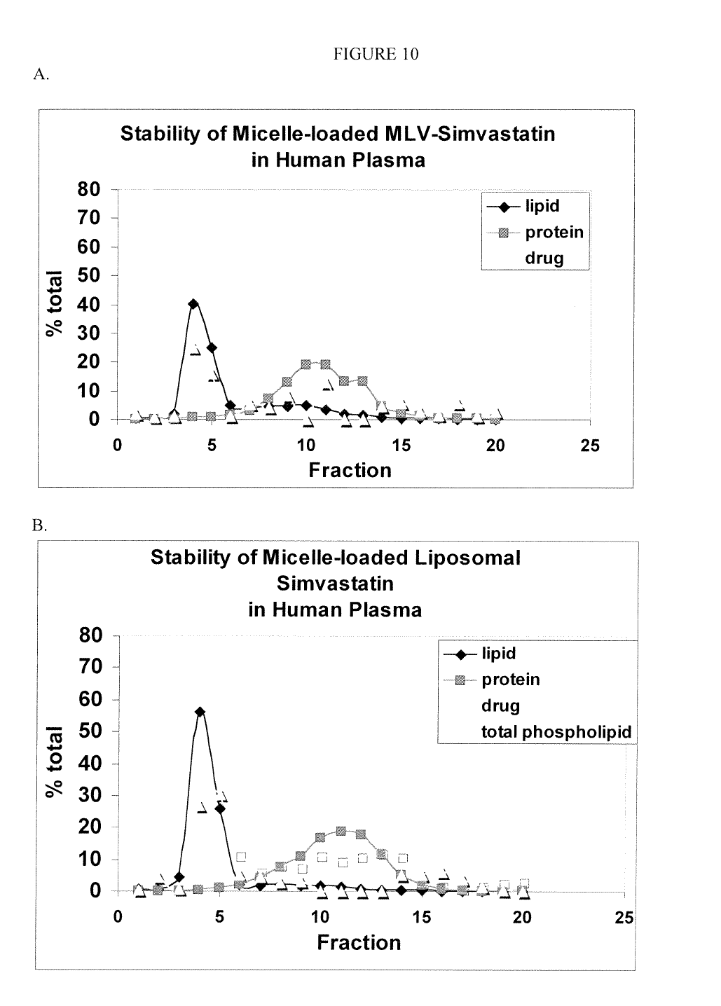

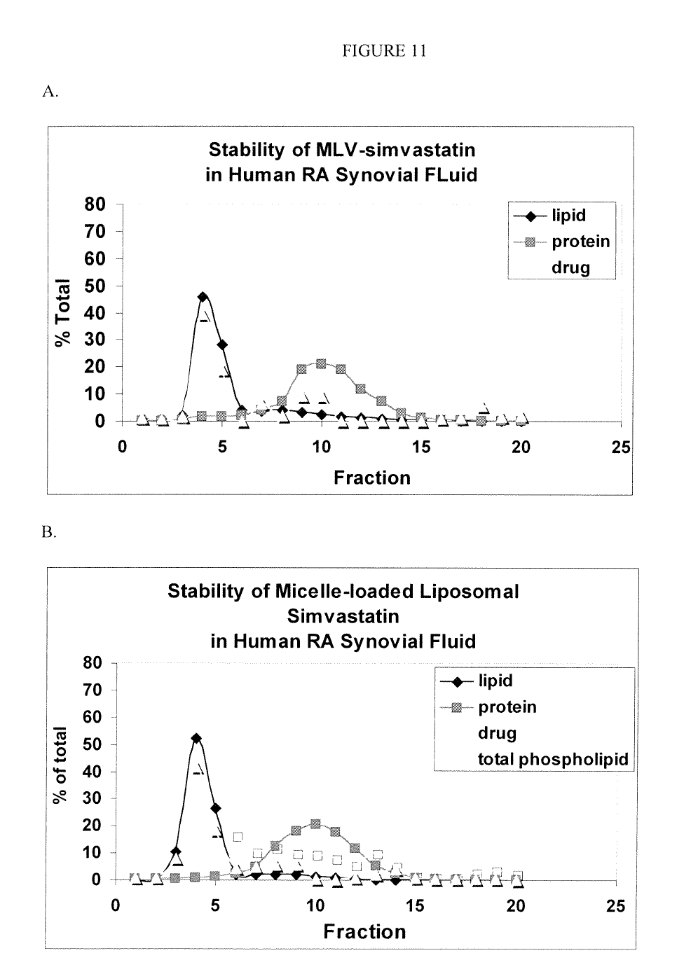

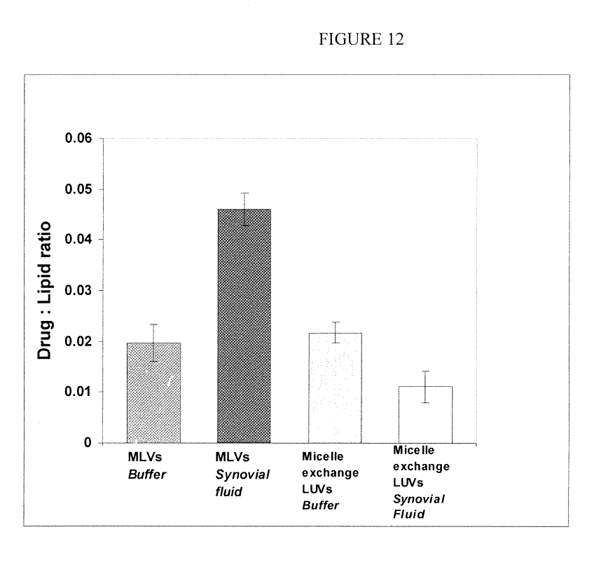

Following incubation in HEPES-buffered saline (HBS), plasma or human synovial fluid from patients with rheumatoid arthritis (RA) for 2 h at 37° C., liposomal samples were passed down a CL4B gel filtration column and fractions were collected to separate the liposomal fractions (2-6) from the micelle and protein fractions (10-15). The results are shown in FIG. 9A (MLV-simvastatin in HBS buffer), 9B (ML liposomal simvastatin in HBS buffer), FIG. 10A (MLV-simvastatin in human plasma), 10B (ML liposomal simvastatin in human plasma), FIG. 11A (MLV-simvastatin in human RA synovial fluid) and 11B (ML liposomal simvastatin in human RA synovial fluid). Data represent mean of 3 sets of independently prepared samples, shown as % of total added to the fractionation column that was recovered in each fraction. The results indicate that simvastatin remains liposome-associated in HBS and only a minor amount separates from the liposomes in plasma or synovial fluid to become associated with lipid micelles or proteins. FIG. 12 presents a bar graph showing that the original drug:lipid ratio of 0.02 (w/w) was not reduced following buffer incubation of MLV-simvastatin and ML liposomal simvastatin and following synovial fluid incubation of MLV-simvastatin and only slightly reduced for synovial fluid incubation of ML liposomal simvastatin.

### EXAMPLE 8

### Effect of Lipid Composition on Stability of Drug Loading for Liposomal Simvastatin

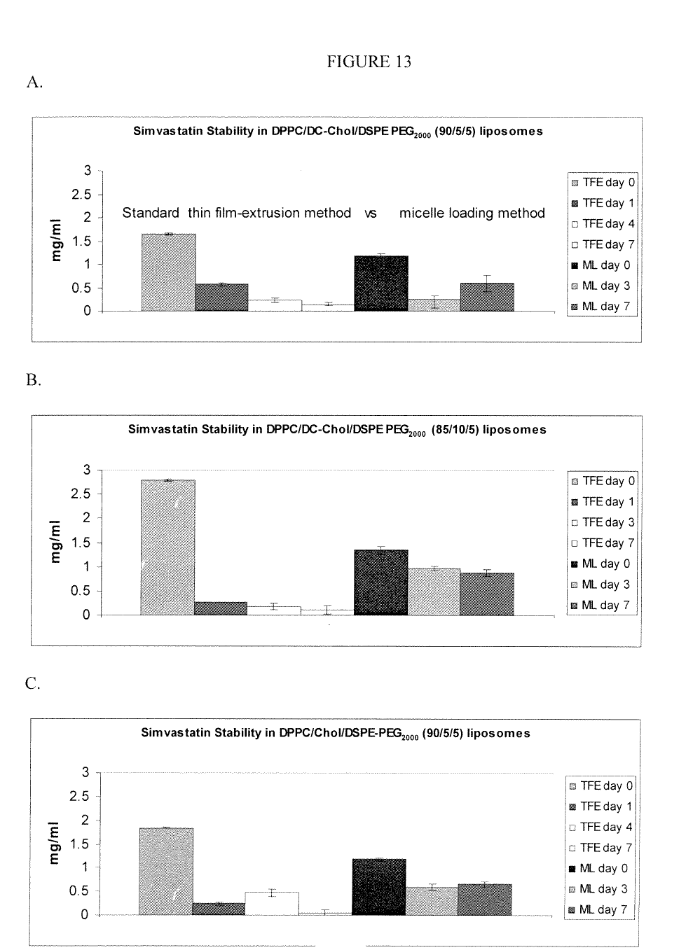

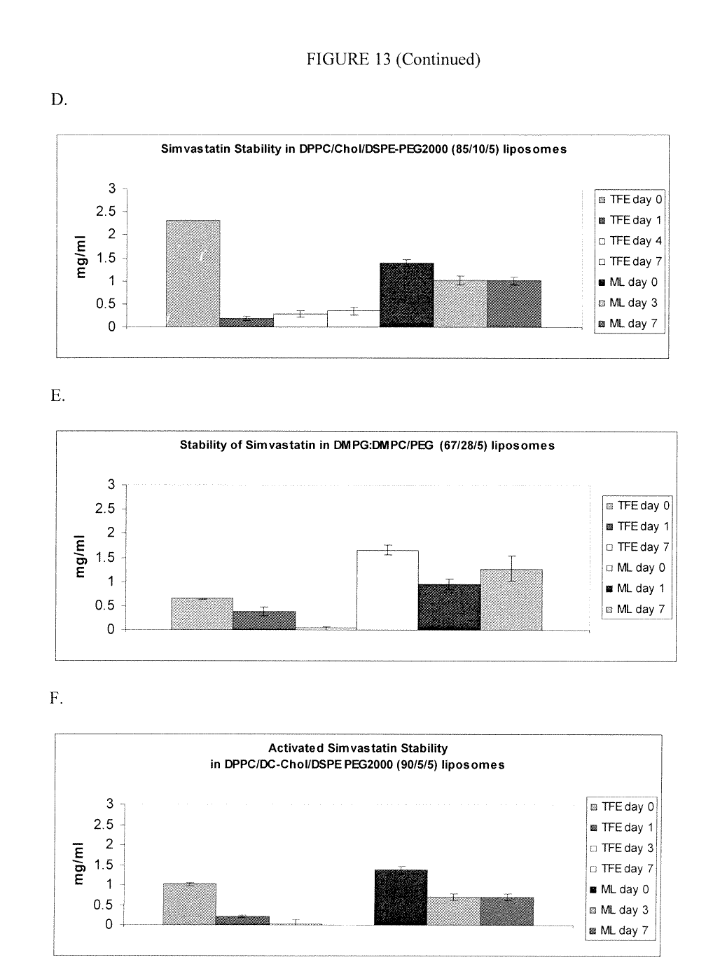

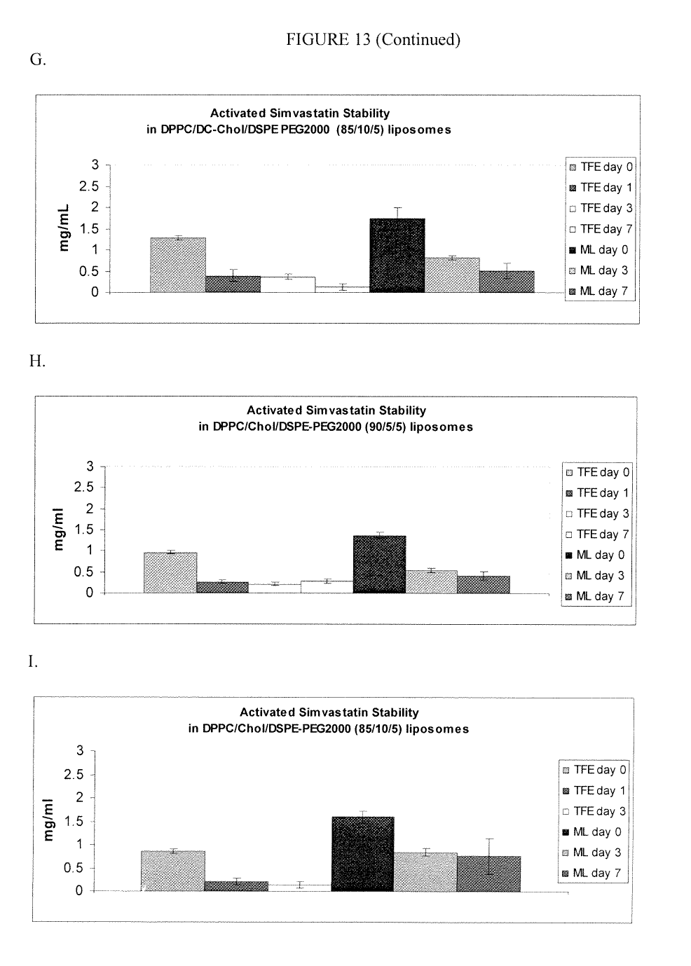

Liposomal simvastatin was prepared by a standard thin-film extrusion method (TFE) and the micelle-loading method (ML). Liposomes were stored in HEPES-buffered saline (pH 7.2) for 1, 3 or 4 and 7 days followed by analysis of drug content in the liposomes. The results are shown in the graphs in FIGS. 13 A-I. Lipid composition is indicated on each graph, with molar ratios of components indicated. DPPC: dipalmitoyl phosphatidylcholine; chol: cholesterol; DSPE-PEG: distearoyl phosphatidylethanolamine-poly(ethylene glycol); DC-chol: 3β-[N-(Dimethylaminoethane)carbamoyl]cholesterol; DMPC: dimyristroyl phosphatidylcholine; DMPG: dimyristoyl phosphatidyl glycerol. Data represent mean ±SD for 3 independently prepared liposomal samples. The micelle loading method results in more stable drug loading for liposomal simvastatin than the standard TFE method regardless of lipid composition, and regardless of whether simvastatin is in the lactone or carboxylic acid form (“activated simvastatin”) indicating the flexibility of this drug loading method.

TABLE 1

| | | | Mean diameter after |
|---|---|---|---|
| Liposomal Phospholipid | | | addition of DSPE-PEG |
| Formulation | Mean diameter | | micelles at 50° C. |
| DPPC | >>1 | um | 152.4 ± 48.8 nm |
| DPPC/econazole | 143.5 ± 45.6 | nm | 165.3 ± 69.2 nm |
| DMPC | 159.4 ± 38.3 | nm | 160.2 ± 52.7 nm |
| DMPC/econazole | 141.2 ± 41.0 | nm | 139.9 ± 46.3 nm |

TABLE 2
Multidose tolerability of liposomal econazole

| | Alkaline | | ALT | AST | Bilirubin | |
|---|---|---|---|---|---|---|
| | phosphatase | GGT | (SGPT) | (SGOT) | (total) | |
| Treatment | (35-200) | (0-1) | (0-50) | (70-900) | (0-7) | Sorbital |
| group | IU/L | IU/L | IU/L | IU/L | μmol/L | Dehydrogenase |
| Empty | 160 ± 10 | 3.7 ± 1.2 | 79.3 ± 69.3 | 135 ± 65 | 5 ± 2 | 50.3 ± 37.3 |
| liposomes | | (↑) 3/3 | | | | |
| Liposomal | 149 ± 12.6 | 3 ± 1.7 | 60 ± 50.5 | 188 ± 88 | 4.3 ± 4.5 | 22.7 ± 13.1 |
| econazole, | | (↑) 2/3 | (↑) 1/3 | | | |
| conventional | | | | | | |
| Liposomal | 175 ± 7 | 3 ± 1 | 37.7 ± 6 | 129 ± 6.6 | 4 ± 2.6 | 26.3 ± 1.9 |
| econazole, | | (↑) 3/3 | | | | |
| micelle- | | | | | | |
| loaded | | | | | | |

TABLE 3
Liver enzyme changes in tumor-bearing mice that received liposomal
econazole in an efficacy study

| | | | | | | |
|---|---|---|---|---|---|---|
| Empty | 215 ± 34.6 | 4.7 ± 0.6 | 53 ± 11.7 | 122 ± 30 | 5.3 ± 1.5 | 33 ± 4.7 |
| liposomes | (↑) 2/3 | (↑) 3/3 | (↑) 2/3 | | | |
| Liposomal | 288 ± 90.7 | 3.7 ± 2.3 | 66 ± 7.6 | 192 ± 56 | 6 ± 4 | 32.6 ± 7.3 |
| econazole, | (↑) 2/3 | (↑) 2/3 | (↑) 3/3 | | (↑) 1/3 | |
| conventional | | | | | | |
| Liposomal | 225 ± 105 | 3.3 ± 3.0 | 36 ± 8.0 | 121 ± 46 | 8.3 ± 0.5 | 25.9 ± 1.6 |
| econazole, | (↑) 1/3 | (↑) 2/3 | | | (↑) 1/3 | |
| micelle-loaded | | | | | | |

TABLE 4
Serum electrolytes and renal function assessment in in tumor-bearing mice
that received liposomal econazole in an efficacy study

| | Na⁺ | K⁺ | Ca²⁺ | Phosphorus | Cl⁻ | CO₂ | BUN | Creatinine |
|---|---|---|---|---|---|---|---|---|
| Treatment | (143-152) | (0-1) | (2.14-2.54) | (1.73-3.51) | (103-117) | (14-28) | (6-17) | (30-56) |
| group | mmol/L | mmol/L | mmol/L | mmol/L | mmol/L | mmol/L | mmol/L | μmol/L |
| Empty | 148 ± 2 | 8.0 ± 0.3 | 2.5 ± 0.03 | 2.5 ± 0.1 | 113 ± 1 | 27.3 ± 1.2 | 6.6 ± 0.5 | 20 ± 3 |
| liposomes | | | | | | | | |
| Liposomal | 153 ± 5.8 | 7.9 ± 0.4 | 2.7 ± 0.2 | 2.4 ± 0.3 | 118 ± 5.9 | 27 ± 1.7 | 8.1 ± 3.8 | 23.3 ± 7.0 |
| econazole, | | | | | | | | |
| conventional | | | | | | | | |
| Liposomal | 150 ± 1.5 | 8.3 ± 0.5 | 2.6 ± 0.1 | 2.5 ± 0.1 | 115 ± 1.5 | 28.3 ± 1.5 | 6.1 ± 0.4 | 22.7 ± 5.0 |
| econazole, | | | | | | | | |
| micelle- | | | | | | | | |
| loaded | | | | | | | | |

What is claimed is:

1. A method for loading an agent into a liposome, the method comprising:
a) combining the agent with a micelle-forming compound to form a micelle comprising the agent, wherein the agent is releasable from said micelle-forming compound; and
b) adding the micelle to the liposome, wherein the micelle combines with the liposome such that the agent is loaded into the liposome to form a loaded liposome.

2. The method of claim 1, wherein in step (b), the micelle combines with the lipid bilayer of the liposome.

3. The method of claim 1, wherein the micelle-forming compound comprises a hydrophilic or amphipathic moiety.

4. The method of claim 3, wherein the micelle-forming compound is a PEG-lipid conjugate.

5. The method of claim 4, wherein the PEG-lipid conjugate is DSPE-PEG2000.

6. The method of claim 1, wherein the agent is dissolved in a solvent.

7. The method of claim 6, wherein the solvent is ethanol.

8. The method of claim 7, wherein the agent is a compound that is poorly soluble.

9. The method of claim 1, wherein the agent is a therapeutic agent.

10. The method of claim 9, wherein therapeutic agent is selected from econazole and pharmaceutically acceptable salts, solvates and prodrugs thereof and mixtures thereof.

11. The method of claim 9, wherein the therapeutic agent is an anticancer agent or an antifungal agent.

12. The method of claim 9, wherein the agent is a statin.

13. The method of claim 12, wherein the statin is selected from simvastatin, atorvastatin, cerivastatin, fluvastatin, lovastatin, mevastatin, pitavastatin, pravastatin, rosuvastin mixtures thereof and pharmaceutically acceptable salts, solvates and prodrugs thereof and mixtures thereof.

14. The method of claim 1, wherein the loaded liposome is about 100 nm to about 200 nm in diameter.

15. The method of claim 1, wherein the loaded liposome is a unilamellar liposome.

16. The method of claim 1, wherein the loaded liposome comprises one or more of a lipid selected from DMPC or DPPC.

17. The method of claim 1, wherein the loaded liposome comprises a targeting agent.

18. A composition produced by the method of claim 1.

19. The composition of claim 18, further comprising a pharmaceutically acceptable carrier.

20. A liposomal composition comprising econazole, wherein the composition is formulated for parenteral delivery.

21. The composition of claim 20, wherein the composition comprises a lipid selected from DMPC or DPPC.

22. The composition of claim 21, wherein the composition comprises DSPE-PEG₂₀₀₀.

23. A liposomal composition comprising a statin, wherein the composition is formulated for parenteral delivery.

24. The composition of claim 25, wherein the statin is selected from simvastatin, atorvastatin, cerivastatin, fluvastatin, lovastatin, mevastatin, pitavastatin, pravastatin and rosuvastin.

25. The composition of claim 24, wherein the composition comprises a lipid selected from DMPC or DPPC.

26. The composition of claim 25, wherein the composition comprises DSPE-PEG₂₀₀₀.

27. A method of treating a cancer or a fungal infection comprising administering the composition of claim 20 to a subject in need thereof.

28. A method of treating a disease or condition selected from dyslipidemia, hypercholesterolemia, hypertriglyceridemia, cardiovascular disease, acute coronary syndrome, experimental autoimmune encephalomyelitis, rheumatoid arthritis, osteoarthritis, transplantation, multiple sclerosis, chronic kidney disease and influenza comprising administering the composition of claim 23 to a subject in need thereof.

29. The method of claim 28, wherein the disease or condition is selected from dyslipidemia, hypercholesterolemia, hypertriglyceridemia, cardiovascular disease, acute coronary syndrome, rheumatoid arthritis and influenza.

30. A method of delivering a therapeutic agent to a cell in a subject in need thereof comprising administering the composition of claim 18 to said subject.

31. A kit for preparing a loaded liposome comprising a first container comprising a therapeutic agent; a second container comprising a micelle-forming compound; and a third container comprising a liposome of the desired composition, together with instructions for combining the contents of the first and second containers to form a micelle comprising the therapeutic agent, and for combining the micelle with the contents of the third container to prepare a loaded liposome.

32. The kit of claim 31, wherein the therapeutic agent is econazole or a statin.

33. The kit of claim 31, wherein the micelle comprises DSPE-PEG₂₀₀₀.

34. The kit of claim 31, wherein the liposome comprises a lipid selected from DMPC or DPPC.
